(19) United States
(12) Patent Application Publication
(10) Pub. No.: US 2025/0067721 A1
(43) Pub. Date: Feb. 27, 2025

(54) METALLIC CLUSTER MODIFIED NANOPORE SENSING FOR PEPTIDE DETECTION

(71) Applicants: Virginia Commonwealth University, Richmond, VA (US); ROWAN UNIVERSITY, Glassboro, NJ (US)

(72) Inventors: Joseph E. Reiner, Richmond, VA (US); Massimo F. Bertino, Richmond, VA (US); Gregory Caputo, Glassboro, NJ (US)

(21) Appl. No.: 18/812,166

(22) Filed: Aug. 22, 2024

Related U.S. Application Data
(60) Provisional application No. 63/534,632, filed on Aug. 25, 2023.

(51) Int. Cl. G01N 33/487; G01N 33/68

(52) U.S. Cl. CPC G01N 33/48721; G01N 33/6815; G01N 33/6893; G01N 2333/31

(57) ABSTRACT

Methods of detecting cysteine-containing peptides are provided. The methods include steps of contacting a resistive-pulse nanopore sensing system with a sample containing cysteine-containing peptides of at least 10 amino acid residues in length, wherein the resistive-pulse nanopore sensing system comprises thiolate-capped gold clusters arranged in a cis side of nanopores and detecting a change in current indicative of exchange between a cysteine-containing peptide and the thiolate-capped gold clusters.

### CROSS-REFERENCE TO RELATED APPLICATIONS

This application claims the benefit of U.S. Provisional Patent Application 63/534,632 filed on Aug. 25, 2023, the complete contents of which are herein incorporated by reference.

### STATEMENT REGARDING FEDERALLY SPONSORED RESEARCH OR DEVELOPMENT

This invention was made with government support under grant number CBET2011173 awarded by the National Science Foundation (NSF). The government has certain rights in the invention.

### SEQUENCE LISTING

This application includes as the Sequence Listing the complete contents of the accompanying XML file “02941700AA_seglisting.xml”, created Aug. 7, 2024, containing 5428 bytes, hereby incorporated by reference.

### FIELD OF THE INVENTION

The invention is generally related to methods for detecting cysteine-containing peptides using a resistive-pulse nanopore sensing system. In particular, the system comprises thiolate-capped gold clusters arranged in a cis side of nanopores.

### BACKGROUND OF THE INVENTION

Peptides play a vital role in various biological processes and their interactions with enzymes and proteins impact critical function.¹⁻³ More specifically, peptides play a significant role in biosignalling, both as primary signalling molecules and effectors after the signal is received.⁴⁻⁷ This makes peptides an important biomarker for various diseases,⁸ which motivates the development of peptide biosensing. Numerous peptide sensing techniques have been explored with many based on either enzyme linked immunosorbent assay (ELISA) tests,⁹ or mass spectrometry (MS).^{10,11} Given the atomic level accuracy enabled by MS, this approach has become the gold-standard analysis tool for identification of peptides. Despite the advantages of these techniques, they are still lacking in availability, cost, and sample throughput thus creating a need for further development of accurate, low-cost, and robust biosensors for the purpose of peptide detection.

Nanopore sensing is a low cost, high throughput, and label free method that has received considerable attention as a possible candidate for peptide and protein detection.¹²⁻¹⁴ This technique has been utilized in small scale, handheld devices like the MinION® system (Oxford Nanopore Technologies) for DNA sequencing and this drives continued efforts to expand the technique to peptide detection. This has led to a variety of successful outcomes in detecting and discriminating select peptides,” but the far more complex problem of sequencing proteins as compared to DNA has resulted in a limited number of protein sequencing demonstrations.¹⁶ Therefore, a need exists to continue advancing the nanopore technique for peptide identification applications.

Improvements to nanopore-based peptide sensing have typically focused on modifications to the pore to yield higher capture rates, interrogation times and/or more clear separation between the signals created by different peptides.¹⁷⁻²¹ Among the many achievements engineered pores have demonstrated is the ability to discriminate between peptides that differ by a single amino acid,²² identification of post translational modifications,^{23,24} and enzyme crosstalk in the renin-angiotensin system.²⁵ In each case, the selectivity and binding can be tuned through nanopore modifications to enhance the pore's ability to capture and discriminate between different target peptides. However, given the large number of peptides that can be present in typical samples, (e.g. a tryptic digest of bovine serum albumin (BSA) yields 74 different peptides) nanopore sensors will struggle with peptide discrimination in real world applications. This issue has been alluded to and addressed through the use of spectral “fingerprinting”,²⁶ but the limited resolution of nanopore sensing would benefit from increased selectivity to reduce the complexity of detecting peptide mixtures.

### SUMMARY

Described herein is the use of gold nanoclusters, trapped in a biological nanopore, enabled for the long time interrogation of cysteine containing peptides. The metallic cluster modified nanopores allow for selective detection of a subpopulation of peptides that can simplify analysis and the extended interrogation times enable more accurate identification of the captured peptide.

An aspect of the disclosure provides a method of detecting cysteine-containing peptides, comprising contacting a resistive-pulse nanopore sensing system with a sample containing cysteine-containing peptides of at least 10 amino acid residues in length, wherein the resistive-pulse nanopore sensing system comprises thiolate-capped gold clusters arranged in a cis side of nanopores; and detecting a change in current indicative of exchange between a cysteine-containing peptide and the thiolate-capped gold clusters.

In some embodiments, the thiolate is tiopronin. In some embodiments, the nanopores are protein nanopores. In some embodiments, the protein nanopores are alpha hemolysin nanopores. In some embodiments, the alpha hemolysin is a wild-type alpha hemolysin. In some embodiments, the sample is a biological sample obtained from a subject. In some embodiments, the biological sample is selected from the group consisting of urine, blood, and saliva. In some embodiments, the subject is suspected of having cancer and the cysteine-containing peptides are biomarkers of the cancer.

### BRIEF DESCRIPTION OF THE DRAWINGS

FIGS. 1A-B. Experimental setup and corresponding peptide attachment traces. (A) Peptide attachment methodology with nanoporebased cluster analysis. (i) A single cluster is loaded into the pore and reduces the current. (ii) Following cluster capture, the target peptide is sprayed onto the trapped cluster and attachment occurs changing the corresponding ligand dynamics. (B) Sample traces illustrate two distinct fluctuation types. (i) A so-called low-frequency fluctuation where several peptides lead to easily discernible current states. (ii) A high-frequency fluctuation where a single peptide yields rapid fluctuations. (iii) A zoomed in view of the resulting ligand dynamics exhibits two-state fluctuations that can be analyzed to identify the target peptide. All data shown here were taken under 70 mV applied transmembrane potential in 3 M KCl at pH 8. The “low-frequency” peptide in panel (B, (i)) is GRGDSPC at 500 μM in the loading tip, and the “high-frequency” peptide in panel (B, (ii)) is 9C1 at 250 μM in the loading tip.

FIGS. 2A-D. Size-dependent high-frequency peptide fluctuations. Peptides anchored at the N-terminus with a cysteine residue yield sizable current fluctuations. Peptides with different mass can be characterized by these fluctuations. (A) Sample current traces and ensemble averaged all-points histograms for 11C1, 9C1, 7C1, and 5C1 yield high-frequency fluctuations upon attachment of a single peptide at 0.1 s. Note that the steady current before capture results from a single trapped TP-capped Au cluster in the nanopore (see FIG. 1). Also note the current distributions are shifted so the upper current state is positioned at zero. We parametrize the fluctuation magnitude with respect to the gold-occupied state (6) and the magnitude of the fluctuations between the upper and lower peptide states (7). The corresponding all-points histograms for each of the peptides show fluctuations yield two discrete states with the relative population of each state dictated by the peptide size. (B) A trend between the peptide mass and “6” fluctuations exist. The solid line is a least-squares weighted-fit forced through the origin with slope (15.5±1.4) fA/(g/mol). (C) Additionally, a trend can be seen for “y” fluctuations. The solid line is a least-squares weighted-fit forced through the origin with slope (79.4±1.6) fA/(g/mol). Note that both linear fits in parts (B) and (C) have been extended to the origin to illustrate the range of linearity. (D) The relative height of the left-most peak scales linearly with the peptide mass for all the peptides and suggests an additional metric (Pleft) for peptide identification. The least-squares weighted fit yields a slope of (1.2±0.1) mmol/g. All data shown here were taken under 70 mV applied transmembrane potential in 3 M KCl at pH 8. The error bars are standard deviations calculated from a minimum of 6 different peptide capture events.

FIGS. 3A-F. Fluctuations depend on the cysteine position, end functional group, and peptide charge. Each current trace shows a single TPcapped gold cluster trapped in the pore for 1 s. At t=1 s, the corresponding peptide is attached to the cluster and various fluctuations ensue. These fluctuations inform about the nature of the peptide/particle interactions. (A, B) The cysteine location in the peptide anchors the peptide to the gold cluster and yields various fluctuations. For the 9C1 peptide, the carboxyl group is furthest away from the core, and this gives rise to well-resolved two-state fluctuations. As the cysteine is positioned near the carboxyl end, the amine group is free to interact with the TP ligands. This gives rise to a more stable and smaller configuration as evidenced by the fluctuations and corresponding all-points histograms. (C, D) Substituting the end functional group from carboxyl (7C1) to amine (7C1-NH2) leads to a more stable and compact conformation. (E, F) Residue charge also affects the fluctuations as seen in the current traces. Negatively charged peptides tend to resist peptide coiling, which in turn yields clearly resolved two-state fluctuations. Net-neutral and positively charged peptides enable more compact cluster structures that eliminate the deeper current state. Note that the all-points histograms correspond to the current traces shown. All data shown here were taken under 70 mV applied transmembrane potential in 3 M KCl at pH 8.

FIGS. 4A-C. Step-based low-frequency peptide sensing. (A) Typical current trace of peptide attachments (this case shows GRGDSPC). The open pore current drops upon entry of a single TP-capped gold cluster at ˜5 s. Free peptide is ejected onto the pore from t=16 to 23 s as indicated with the bar. This yields clearly delineated steps in the current. The overlaid trace shows the current filtered with a 100 Hz low-pass filter. (B) An all-points histogram of the attachment process (t=20-35 s, region highlighted with box) shows discrete current states. The inset shows the linearity of the peak positions, and the solid line is a least-squares fit whose slope is equal to the current step magnitude for that particular peptide. (C) The peak steps are nearly linear up to 1000 g/mol. The solid line is a least-squares fit with a slope value of (13.4±0.4) fA/(g/mol), which is in reasonable agreement with the slope found in FIG. 2B. The ligands and peptides shown here listed in order of increasing mass are (TP, glutathione, RGDC, 5C1, GRGDSPC, and 9C7). All data shown here were collected under 70 mV applied transmembrane potential in 3 M KCl pH 8 buffer. Each data point corresponds to the average slope calculated from a minimum of 4 different cluster experiments. The error bars correspond to ±1 SD.

FIGS. 5A-C. Low-frequency peptide mixture analysis allows discrimination between different-sized peptides by step-size comparison. (A) A typical current trace shows the capture of a TP-capped cluster at t=2.6 s followed by continuous peptide spray onto the pore from t=14 s-27 s. The signal is filtered at 100 Hz and a zoomed-in view of the sub region in blue from t=22-27 s is shown in panel (C). (B) A distribution of 237 down steps (magnitude >4 pA) collected from 3 different experiments (24 clusters on 3 nanopores) superimposed on the corresponding distributions for each peptide individually. The distribution shows a greater frequency of RGDC fluctuations. The peak of each distribution is best fit to the mixture data. (C) Clearly defined state transitions can be used to distinguish between the two peptides in the mixture. This shows a subset of the current trace. Step 2=9.95 pA (GRGRDSPC with 98% probability), step 3=9.58 pA (GRGRDSPC 95%), step 5=7.24 pA (RGDC 99%), step 6=7.32 pA (RGDC 99%), step 7=12.06 pA (GRGDSPC 99%), and step 8=5.04 pA (RGDC 99%). Steps 1 and 4 are on the order of 2 pA and most likely correspond to TP fluctuations.

FIGS. 6A-C. Multistep and mixture analysis for 7C1 and 9C1 peptides. Peptide addition can be clearly resolved with 100 Hz filtered steps (traces) and corresponding histograms can be analyzed to identify attaching peptides. (A, left) Three consecutive 7C1 peptides attach to a single cluster giving rise to (A, right) distinct all-point current histograms. Pleft data (see FIG. 2) shows reasonable agreement with a simple model. (B) Similar behavior can be observed for 9C1 peptides where Pleft data show excellent agreement with a simplified model. (C) A 1:3 molar mixture of 7C1 and 9C1 peptides shows the addition of both peptide types on a single cluster. This particular current trace shows the addition of two 7C1 peptides, followed by two 9C1 peptides. The data overlap with the 7C1 model for peptides 1 and 2 and then agrees with the addition of 9C1 for peptides 3 and 4.

FIGS. 7A-C. Cluster-enhanced cysteine-selective nanopore sensing. (A, top) The multi-step process begins with a peptide-containing and cluster-containing nanopipette tip positioned in the vicinity of an isolated nanopore. Clusters are ejected into the pore until one is captured. (A, bottom) The cluster tip is removed and peptides are ejected towards the cluster-containing pore. (B) Two types of fluctuations ensue. (Top) For smaller peptides, the clusters attach or detach on a seconds timescale while (bottom) larger peptides show a single peptide attachment with subsequent high frequency fluctuations. (C) Typical current traces for these two event types show (top) attachment of smaller peptides or (bottom) or single large peptide. The smaller peptide trace is overlaid with a 100-Hz low-pass filtered signal that clearly demonstrates the discrete attachment steps. In both current traces, transitions from the open pore state (i˜200 pA) to the cluster occupied state (i−145 pA) precede peptide detection. The lower trace shows an inset corresponding to the box around t=10 s that illustrates the complex nature of the resulting fluctuations.

FIGS. 8A-D. Cancer marker peptides exhibit low frequency step-wise fluctuations that correlate with peptide mass. (A) Sample current trace for P9C6 peptide. The raw trace with the overlaid filtered data (100 Hz low pass) which exhibits clear steps between the numbered states. (B) A current histogram of the filtered data yields seven distinct peaks, which are labelled with the numbers in panel (A). The transitions from states 7 to 5, 6 to 3 and 4 to 1 yield three current steps plotted in panel (C). (D) A summary of all cancer marker peptides yields a similar, but more scattered trend than previously analyzed control peptides. Each data point in panel (D) corresponds to the mean and standard deviation measured from the following number of steps and pores respectively: P8C5=(6, 2), P9C6=(6, 1), P9C9=(3, 1), P9C6FC=(8, 3), P9C9FC=(11, 2), P16C1FT=(6, 2), P16C9FT=(8, 1) and P16C15FT=(9, 2). The cancer marker peptide data in panel (D) are least-squares-fit with a linear function and fixed origin to a slope of (16.6+/−0.5) fA/(g/mol), which is slightly larger than the least-squares-fit to the previously analyzed synthetic peptides (13.4+/−0.4) fA/(g/mol).¹⁷

FIGS. 9A-F. Cluster-based high-frequency fluctuation analysis of different sized cancer marker peptides yields more accurate discrimination than open pore analysis. Panels (A-C) correspond to open pore analysis. Panels (D-F) correspond to the cluster-based analysis. (A) Sample current traces of open pore analysis show various current blockades corresponding to the different peptides analyzed. (B) Scatter plot of the blockade time and normalized blockade depth for 500 blockade events from each peptide. (B, inset) A sample current blockade with the open pore current (io), blockade current (i) and blockade time (tB). (C) Overlap percentage matrix for peptide pairs shows ˜25% overlap between each peptide. The non-unity values along the diagonal result from the fact that the overlap calculation was confined to the axes range shown (0<tB<20 ms and 0<i/io<0.5) and not all blockade events fall within these boundaries. (D) Sample traces and corresponding all-points histograms from each peptide attached to a pore-bound cluster. Curves in the histograms correspond to the average histogram from a minimum of 5 cluster events. Individual cluster events shown as traces. (E) Normalized autocorrelation for each peptide, fine curves are individual events and the bold lines show each average). (E, inset) Least squares fits of the averaged ACF with single exponential functions (Aexp(−(t/τmean)), show that the mean correlation times scale exponentially with the peptide mass. (F) Probability matrix for identifying a measured peptide (down a column) when compared against the averaged histogram and correlation time (along rows) (see data analysis subsection). For example, the probability of detecting a P16C1 peptide and identifying it as a P13C1 peptide is 14.5%.

FIGS. 10A-H. High frequency fluctuation results from similar-mass 16-mer cancer marker peptides show the superiority of the gold cluster approach compared to open pore analysis for discriminating between peptides (P16C1 SEQ ID NO: 1; P16C4 SEQ ID NO: 2; P16C9 SEQ ID NO: 3; P16C12 SEQ ID NO: 4; and P16C15 SEQ ID NO: 5). The single cysteine residue is positioned at different positions within each peptide sequence. (A-E, left) shows cluster-based sample current traces for each peptide type with (A-E, middle) corresponding all-points histograms of the peptide-induced fluctuations. (A-E, right) Corresponding autocorrelation functions for each trace averaged over a minimum of 5 different cluster captures yields distinct correlation time fluctuations. (F) The probability of incorrectly identifying a measured peptide (along a column) against the average histogram and correlation time (along the row) is well below 5% and near zero in almost all cases. The only significant exception is the 6.2% probability of identifying a P16C9 peptide as a P16C1 peptide. (G-H) Open pore analysis shows far greater overlap in the scatter distribution as expected for equal sized peptides.

FIGS. 11A-E. The only two cysteine-containing peptides from the LRG-1 protein that present in the urine of ovarian cancer patients (P17C2 and P19C2) yield clearly detectable fluctuations in the nanocluster-based sensor. (A) Typical current traces show a single gold nanocluster from t=0-0.4 s when a single peptide is captured. This yields clear fluctuations and a zoomed in view between t=1.4 s-1.7 s shows easily discernible differences in the kinetics. The larger peptide (P19C2) remains in the high current state for longer periods of time. (B) Averaged all-points histograms of the current fluctuations (bold traces) along with the 8 different distributions for each peptide from which the averages are calculated. (C) Autocorrelation analysis shows differences in the kinetics for each peptide, and least squares fits to each correlation trace yield mean correlation times for the two peptide types τP17C2=(0.77+/−0.56) ms and τP19C2=(2.7+/−0.9) ms. (D) Open pore scatter plots show a high degree of overlap. (E) Comparison of the separation probability matrix for cluster analysis and the open overlap distributions show greatly enhanced selectivity for cluster-based analysis (<10% for cluster analysis and 36% for open pore analysis).

FIGS. 12A-C. Demonstration of P19C2 detection in urine demonstrates the feasibility of working in bodily fluids. (A) Sample current traces, (B) autocorrelation analysis, and (C) histogram. The correlation time from a least squares fit of the ACF is 1.8 ms which is within the expected value reported in clean buffer.

### DETAILED DESCRIPTION

Embodiments of the disclosure provide methods for cysteine-selective peptide detection using gold cluster-enhanced nanopores. By temporarily introducing a gold nanocluster into the cis-side of a nanopore, it is shown herein that peptides containing cysteine residues can be captured and analyzed for extended periods. Cysteine is the least abundant amino acid in the proteome (<2% of all amino acids in the UniProt TREMBL data base are cysteines) making it an ideal target for reducing peptide mixture complexity. This cysteine selectivity reduces the complexity of peptide-based sensing by eliminating many of the peptides from a sample that may complicate data analysis and interpretation. In addition to this reduced complexity, the cluster serves to greatly extend the peptide's interaction time with the pore and thus improve the ability of the nanopore sensor to correctly identify a captured peptide.

Nanopore sensing is an important technique for single molecule analysis for two major reasons: i) It enables label free detection of a wide variety of biomolecules and ii) It is based on electronic measurements so it does not require large and expensive hardware. The use of nanopore sensing for detection of cysteine-containing peptides has not been previously described. The principle of operation for resistive-pulse nanopore sensing is based on the Coulter counter where a nanosized hole in a membrane partition is used to detect single molecules that enter the hole and partially block the flow of current. These blockages can be analyzed to ascertain properties of the molecule in question. Embodiments of the present disclosure utilize modifications to the nanopore environment to facilitate long-time extended interrogation of single molecules. Such modifications (e.g. thiolate-capped gold clusters arranged in a cis side of nanopores) have the advantage of enabling clear identification of the molecule of interest without the need for constructing current blockade or transit time distributions.

A “cluster” may refer to a single nanoparticle. Typically, these clusters comprise about 50-100 gold atoms and about 20-30 ligand molecules that passivate the cluster surface. In some embodiments, a single cluster (nanoparticle) fits in the nanopore.

The term “nanopore,” as used herein, generally refers to a pore, channel or passage formed or otherwise provided in a membrane. A nanopore can be defined by a molecule (e.g., protein) in a membrane. A membrane can be an organic membrane, such as a lipid bilayer, or a synthetic membrane, such as a membrane formed of a polymeric material. The nanopore may be disposed adjacent or in proximity to a sensing circuit, such as, for example, a complementary metal-oxide semiconductor (CMOS) or field effect transistor (FET) circuit. A nanopore may have a characteristic width or diameter on the order of 0.1 nanometers (nm) to about 1000 nm, e.g. about 1-3 nm. Some nanopores are proteins. Alpha hemolysin is an example of a protein nanopore. In some embodiments, the nanopore comprises a wild-type protein nanopore and is not a mutated protein nanopore. In some embodiments, the nanopore is a mutated protein nanopore. Other suitable biological nanopores include, but are not limited to, Aerolysin (AeL),

Mycobacterium smegmatis porin A (MspA), Curlin sigma S-dependent growth subunit G (CsgG), actinoporin fragaceatoxin C (FraC), Outer membrane protein G (Omp G), and Cytolysin A (CyA).

An α-hemolysin protein is a monomeric polypeptide which self-assembles in a lipid bilayer membrane to form a heptameric pore, with a 2.6 nm-diameter vestibule and 1.5 nm-diameter limiting aperture (the narrowest point of the pore) (Meller et al. 2000, Akeson et al. 1999, and Deamer et al. 2002). In an aqueous ionic salt solution such as KCl, when an appropriate voltage is applied across the membrane, the pore formed by an α-hemolysin channel conducts a sufficiently strong and steady ionic current. When peptide ligands undergo exchange and/or additions with the thiolate-capped gold clusters described herein, they give rise to easily resolved current steps. Herein, the term “attachment” is used to describe either a peptide exchange event or direct addition to the pore-bound cluster surface. Attachment can block or reduce the ionic current that is otherwise unimpeded. A particular peptide, when attaching to the gold clusters generates a characteristic signature that distinguishes it from other peptides.

Some embodiments of the disclosure involve steps of measuring of electrical current or an equivalent electrical parameter as a function of time between two liquid compartments. The liquid compartments are separated by and fluidically coupled through a nanopore. The electrical current or equivalent electrical parameter is measured upon attachment of a cysteine-containing peptide.

In various embodiments, the method involves measuring conductivity, resistivity, resistance, conductance, current flow, voltage, current distribution, mean correlation times, or other electrical parameters measured between the two compartments. In a typical embodiment, current is measured. Thus, in various embodiments, the method involves maintaining a voltage between an anode in one compartment and a cathode in the other compartment, and measuring the current over time as perturbations arising from the attachment of cysteine-containing peptides to the nanopore.

Parameters determined with the methods described herein can be used to identify or quantify cysteine-containing peptides in a test composition. For example, if known peptides are present in a solution, the parameters can be determined as the peptides attach to the nanopore, and the values of those parameters are used to identify the peptides.

In another embodiment the method permits the simultaneous determination and/or quantification (i.e. measuring the relative concentration) of various peptides in a test solution. In another embodiment, the method further comprises quantifying the relative number of the individual peptides in the solution.

A suitable device includes a first liquid compartment, a second liquid compartment, and a nanopore disposed between the compartments. The nanopore defines a fluid conduit between the first liquid compartment and the second liquid compartment. The device also includes electrodes in both liquid compartments and means for controlling the electrodes to measure electrical resistance, voltage difference, or ionic current flow between the first and second electrodes. In some embodiments, the device is a hand-held device. In some embodiments, the method is incorporated into a parallel setup (i.e. many nanopores detecting peptides at once), which allows rapid detection of a large number of different peptides. In some embodiments, the method is applied to a large number of nanopores in parallel operation, e.g. 100-5000 nanopores or more.

The thiolate-capped gold nanoparticles arranged in a cis side of nanopores enable selective detection of cysteine-containing peptides while non-cysteine containing peptides are not detected. In some embodiments, the thiolate is tiopronin. Other suitable thiolates include, but are not limited to, glutathione, 4-mercaptobenzoic acid, 3-mercaptobenzoic acid, 2-mercaptobenzoic acid, thiolated PEG, cysteine, methionine, thiomalate, 1-thio-beta-D-glucose, and short (e.g. 2-4 amino acid residues in length) water-soluble peptides that contain a single end cysteine residue. In some embodiments, the ligand molecules are any water soluble thiolate that can passivate and stabilize gold nanoparticles in an aqueous environment.

The approach allows the peptide to remain on the sensor for tens of seconds, and this improves the accuracy of peptide detection over more traditional nanopore sensors. The peptides detected by the methods described herein are at least 10 amino acids in length, e.g. 10-100 amino acids in length, e.g. 10-50 or 10-25 amino acids in length. In some embodiments, the peptides contain 1 or more cysteine residues. In some embodiments, the peptides may correspond to biomarkers or fragments thereof of various diseases, disorders, or conditions. In some embodiments, the peptides are cancer biomarkers. In some embodiments, the methods described herein serve as an initial check for various peptide markers and if some are found, it will function as justification for more thorough analysis via mass spectrometry, which is not readily available outside large urban or university hospitals.

The methods described herein may be used to detect and distinguish cysteine-containing peptides in a solution, e.g. a biological sample. In some embodiments, the biological sample is, or is derived from, urine, blood, or saliva.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Other than in the operating examples, or where otherwise indicated, all numbers expressing quantities of ingredients or reaction conditions used herein should be understood as modified in all instances by the term “about.” The term “about” is used herein to mean approximately, roughly, around, or in the region of. When the term “about” is used in conjunction with a numerical range, it modifies that range by extending the boundaries above and below the numerical values set forth. The term “about” when used in connection with percentages will mean.+−0.1%.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### Example 1. Selective Detection and Characterization of Small Cysteine-Containing Peptides with Cluster-Modified Nanopore Sensing

### Abstract

One can monitor ligand-induced structure fluctuations of individual thiolate-capped gold nanoclusters using resistive-pulse nanopore sensing. The magnitude of the fluctuations scales with the size of the capping ligand, and one can observe ligand exchange in this nanopore setup. Here, we explore the different types of current fluctuations associated with peptide ligands attaching to tiopronin-capped gold nanoclusters. We show here that the fluctuations can be used to identify the attaching peptide through either the magnitude of the peptide-induced current jumps or the onset of high-frequency current fluctuations. Importantly, the peptide attachment process requires that the peptide contains a cysteine residue. This suggests that nanopore-based monitoring of peptide attachments with thiolate-capped clusters could provide a means for selective detection of cysteine-containing peptides. Finally, we demonstrate the cluster-based protocol with various peptide mixtures to show that one can identify more than one cysteine-containing peptide in a mixture.

### Results and Discussion

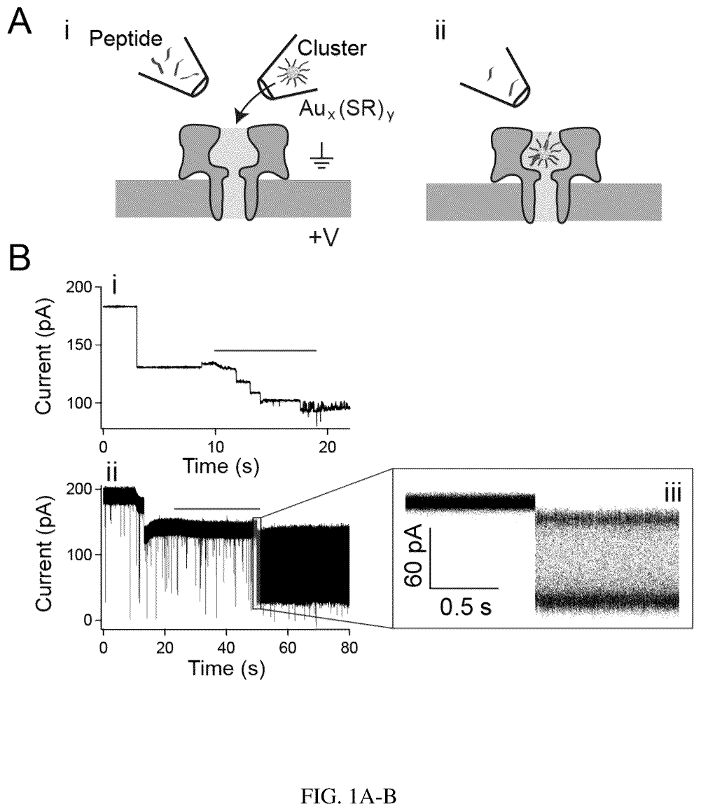

Nanopore analysis can be used in conjunction with microcapillary tips to load individual metallic clusters into a pore.^{56,57} This has been used to study ligand exchange processes on metallic clusters,⁵¹ and we use this methodology here to further explore peptide induced current fluctuations. FIG. 1 illustrates the experimental setup and typical traces that demonstrate peptide attachment. The sensor comprises a wild-type alpha hemolysin (αHL) pore, which is a stable, transmembrane, heptameric pore-forming toxin from Staphylococcus aureus. This αHL pore is loaded with a single TPcapped gold cluster locally injected into the cis-side of the pore. Single particle capture is most likely (rather than multiple particle capture) because the TP ligands are fully deprotonated in the pH 8 condition studied throughout, which makes the clusters charged and prone to cluster—cluster repulsion. Further discussion of this point can be found in the Methods section and elsewhere.⁵¹ Upon capture of a single cluster, pressure applied to the microcapillary containing clusters is turned off to further reduce the likelihood of multiple cluster captures. After a short delay (ca. seconds), solution from a second capillary, containing peptide ligands, is ejected onto the pore and this yields peptide attachment events.

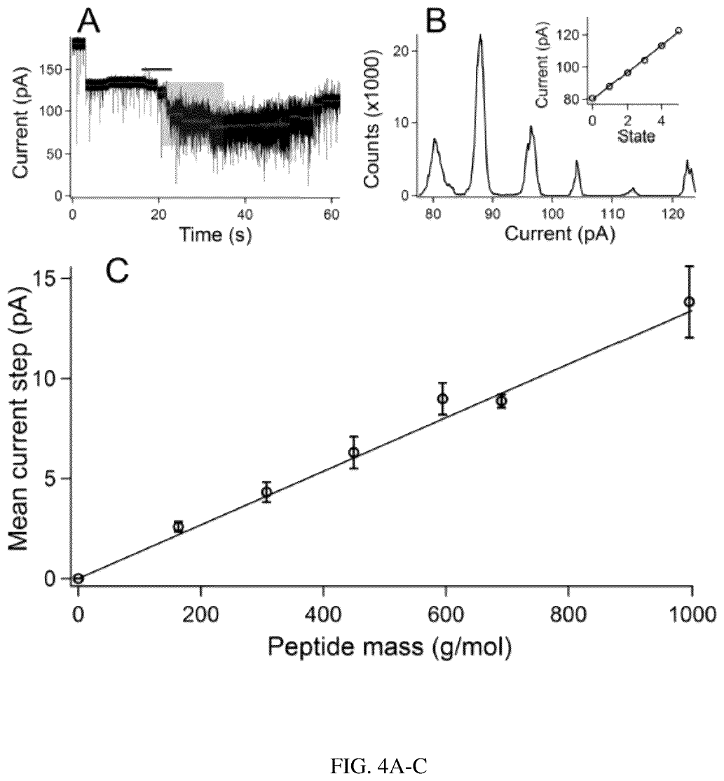

To facilitate the design of a cluster-modified nanopore peptide sensor, we experimented with numerous cysteine-containing model peptides varying the mass, primary sequence, charge, and terminal capping group to better understand the types of fluctuations that result from peptide-cluster interactions. Previous studies showed that when glutathione and thiolated polyethylene glycol (S-PEG) ligands undergo exchange and/or additions with TP-capped clusters, they give rise to easily resolved current steps that can be used to estimate the mass of the attaching ligand.⁵¹ Note that herein we use the term “attachment” to describe either a peptide exchange event or direct addition to the pore-bound cluster surface. See FIG. 4 in ref 51 for a more complete discussion of the differences between ligand exchange and ligand additions. Interestingly, we find that attachment only works for thiol-containing ligands. Consequentially, we find for the case of peptide ligands, they must contain a cysteine residue in order to initiate the attachment process. This is illustrated by numerous current traces comparing attachment (or lack thereof) for cysteine and noncysteine containing peptides.

In this study we find evidence of current stepwise transitions for some peptides (i.e., FIG. 1B,i), but we also find a different type of interaction where the attachment of a peptide ligand yields high-frequency one- or two-state fluctuations (FIG. 1B,ii,iii). We start with a series of experiments to better understand the underlying mechanisms that give rise to the so-called high-frequency current fluctuations.

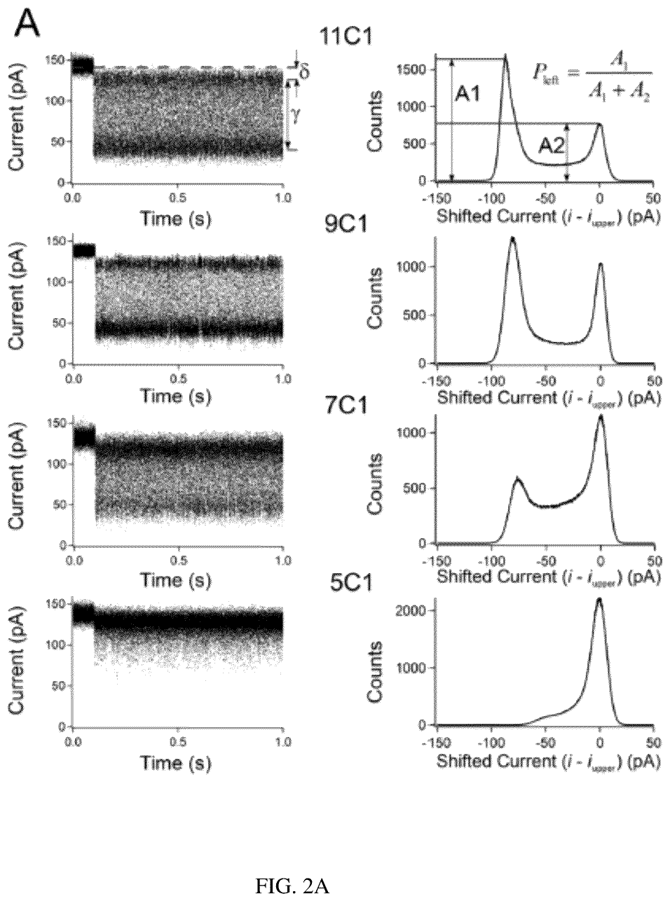

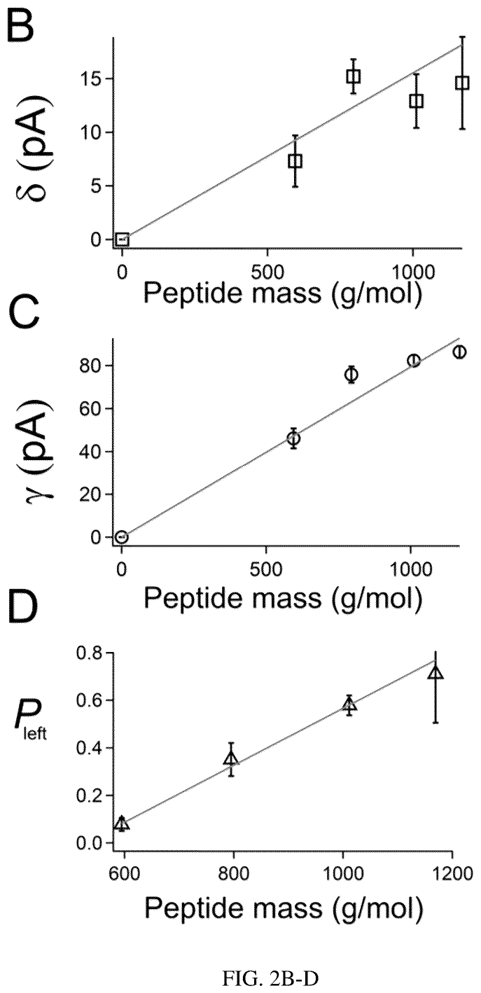

The first experiment, highlighted in FIG. 2, explores the dependence between the high-frequency two-state fluctuations and peptide size. We quantify differences between the peptides by measuring the difference between the TP-capped cluster level and the upper current state (defined as δ=iTP−iupper) and the difference between the upper and lower current states (defined as γ=iupper−ilower). We also find a clear dependence between peptide size and relative occupation of the lower or deeper current state. This is parametrized herein as Pleft=A1/(A1+A2), where A1 and A2 are the heights of the two peaks in the all-points histograms for the peptide occupied current fluctuations (see FIG. 2 for details). FIG. 2 shows a dependence between the attaching peptide mass and δ, γ, and Pleft. This dependence is expected for δ and γ because the difference between the TP-capped cluster before and after the attachment of a single peptide should reduce the current (proportional to δ), and the transitions between the upper and lower current levels likely result from the attached peptide fluctuating near the cluster surface (proportional to γ). However, the dependence between mass and Pleft is less obvious, but it most likely results from the fact that the larger peptides are more prone to blocking the current for longer periods. Regardless of the mechanism driving the Pleftdependence, we find a clear linear trend over the range studied. Taken together, these results suggest that differences between current fluctuations could be used to elucidate the identity of an attaching peptide. However, it is not clear what peptide properties give rise to two-state versus one-state highfrequency fluctuations or low-frequency fluctuations. This uncertainty complicates the development of the clusteroccupied pore as a sensor. To address this, we performed a series of experiments to better understand the mechanisms behind the various high-frequency fluctuations.

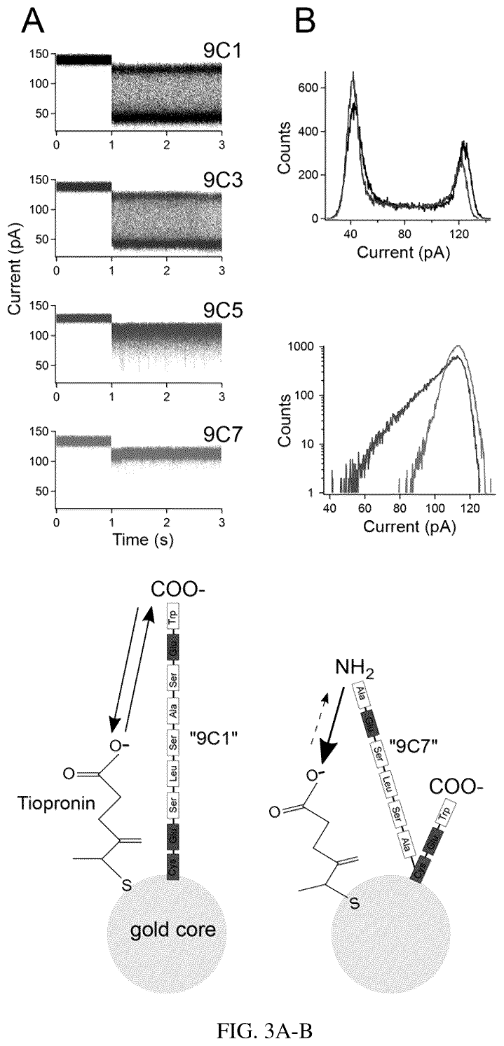

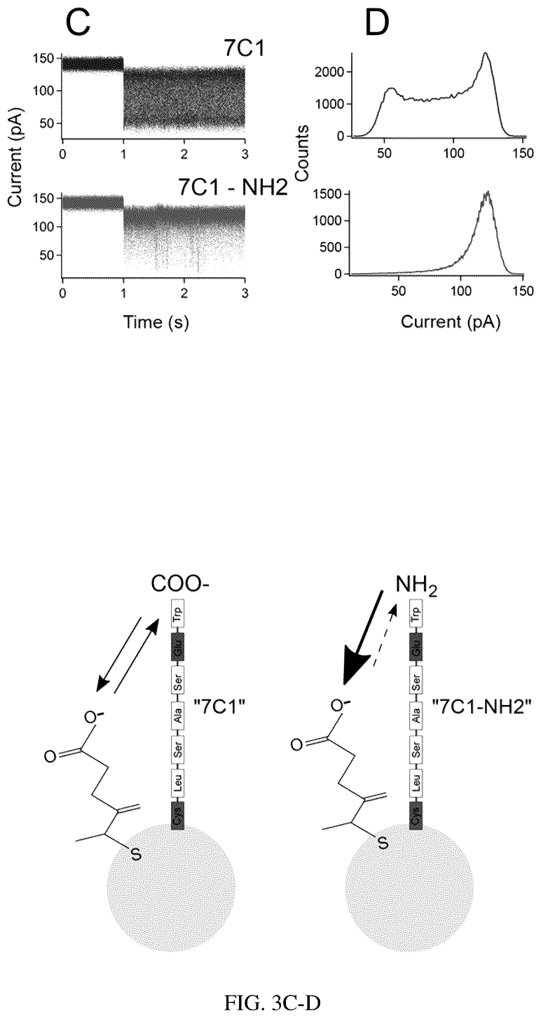

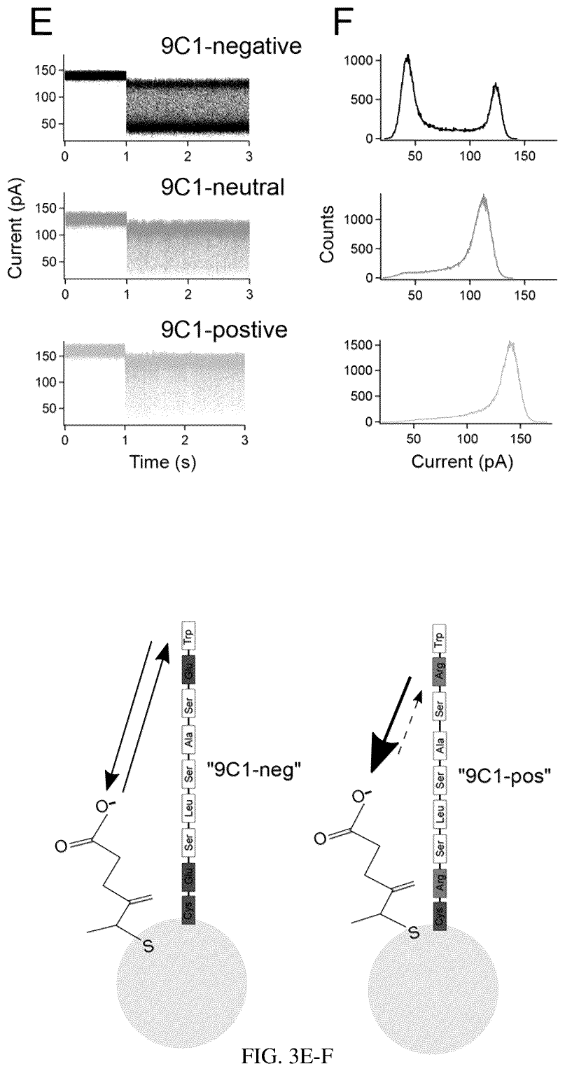

FIG. 3 shows the role that cysteine position, peptide charge, and capping functional group play on the highfrequency fluctuations. FIG. 3A,B shows sample current traces and corresponding all-points histograms for four different peptides. Each peptide has nine amino acid residues, with the lone cysteine residue located at different positions within each sequence (i.e., 9C1, 9C3, 9C5, and 9C7). Moving the cysteine residue further from the N-terminus yields twostate current fluctuations with diminishing occupation of the deeper current state. We hypothesize that this behavior results from the interactions between the carboxyl group on the TP ligands and the C- or N-termini of the peptide. Specifically, we believe the attaching peptide tends to be in either one of two structural configurations. One corresponds to the peptide being stretched out away from the cluster (larger volume yielding the lower current state), which we will refer to here as the “extended state”, and the other configuration corresponds to the peptide being more closely associated with the cluster surface (smaller volume yielding the upper current state). For simplicity, we will refer to this configuration as the “collapsed state”. The amine-carboxyl interaction (N-C) is most likely attractive, while the carboxyl-carboxyl interaction (C-C) is most likely repulsive at pH 8.58 This means that when the peptide end farther from the cluster is a carboxyl group (i.e., 9C1 and 9C3), the repulsive C-C interaction dominates and leads to rapid and clear fluctuations between the two structural configurations. Conversely, when the peptide N-terminus extends further away from the cluster surface (i.e., 9C5 and 9C7), the more attractive N-C interaction and/or entropic component of the peptide's free energy tends to favor the collapsed state and this creates a smaller volume structure, which leads to greater occupation of the higher current state. This is illustrated in FIG. 3A,B.

To further confirm this hypothesis, we modified the end functional group of a peptide and compared the corresponding current fluctuations. FIG. 3C,D shows typical current traces and corresponding all-points histograms for both 7C1 and 7C1-NH2 peptides. The 7C1-NH2 peptides have the standard C-terminal carboxyl group modified with an amine capping group. The resulting traces show that the addition of the N terminus-like moiety on the freely exposed end of the clusterbound peptide nearly eliminates the lower current state. This is consistent with a model that claims the N-C interaction would lead to a more tightly bound peptide-cluster configuration, while the C-C interaction, expected for the 7C1 peptide, yields rapid transitions between the collapsed and extended state configurations. FIG. 3C,D illustrates this behavior.

In addition to the role that the end functional group plays in driving these high-frequency fluctuations, we also hypothesize that charged residues can affect the two-state fluctuations as well. To confirm this, we performed measurements with positive, negative, and net-neutral versions of the 9C1 peptide. FIG. 3E,F shows current traces and histograms of these three peptides where the 9C1 negative peptide yields clearly resolved two-state fluctuations, while the net neutral and positive peptides essentially eliminate these fluctuations. We hypothesize that in this case, the charged residues interact with the deprotonated carboxyl residue of the TP ligands (measurements performed at pH 8), and this yields repulsive interactions for the negatively charged peptide and attractive interactions for the positive peptide. Once again the attractive interaction favors the collapsed state configuration, which leads to a single, higher-level current state for these peptide-particle pairs. FIG. 3E,F illustrates this behavior.

The experiments outlined in FIGS. 2 and 3 lead to several conclusions regarding the high frequency fluctuations. First, longer peptides are more likely to exhibit two-state fluctuations. Second, the location of the cysteine residue with respect to the C- or N-terminus plays an important role in predicting the onset of two-state fluctuations. Specifically, peptides having the cysteine near the N-terminus are more likely to yield two-state fluctuations. Third, the overall net charge of the peptide also plays a role in the onset of two-state fluctuations. Positive charges tend to create smaller and more stable clusters that eliminate the two-state fluctuations.

It is important to understand the mechanisms that drive these fluctuations because the goal here is to utilize peptide attachment with gold clusters to identify peptides. Given the complex nature of the induced fluctuations from peptide attachment, knowing the connections between peptide sequence and fluctuation type will help with the development of this protocol for peptide sensing. It is worth noting that we have assumed the observed fluctuations reported in FIG. 3 result from interactions between the peptides and the carboxyl group of the TP. It is possible that there may be interactions with the nanopore as well. We believe this is less likely given the fact that the cis-side of the αHL pore has a roughly equal mixture of positive and negative residues, but we have not definitively ruled this out here.

We begin the sensor discussion by exploring peptide analysis of discrete current steps. Developing the cluster-nanopore sensor for detecting and identifying peptides from clearly resolved current steps requires a calibration curve relating current step size to peptide mass. FIG. 4 shows a typical current trace for one such peptide (GRGDSPC). We believe this peptide gives rise to clear current steps as opposed to high frequency two-state fluctuations because of the low mass and the location of the cysteine residue (see FIGS. 2 and 3A,B). This peptide yields clearly resolved current states that can be observed in the 100 Hz low-pass filtered signal (FIG. 4A). An all-points histogram of the filtered signal yields six well-resolved peaks that can be used to identify the different current levels associated with the attachment of this peptide (FIG. 4B and inset). The slope of the inset plot in FIG. 4B yields the current step magnitude for this peptide. We performed a similar step analysis for a variety of different peptides and ligands, and FIG. 4C shows the resulting linear dependence between the mean current step size and the ligand mass for these examples where the mass ranges between ca. 200 and 1000 g/mol.

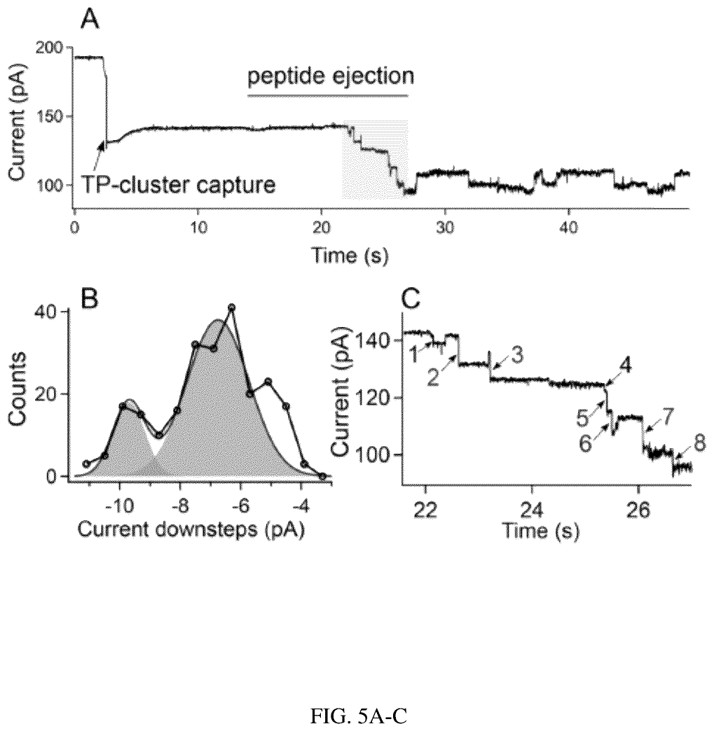

The well-resolved current steps in FIG. 4 suggest that it should be possible to identify peptides from a mixture. We illustrate proof-of-concept of this in FIG. 5, which shows a sample current trace (100 Hz low-pass filtered) resulting from the ejection of a 1:1 mixture (500 μM concentration in the pipet tips for each peptide) of RGDC and GRGDSPC peptides onto a nanopore-bound gold cluster. The ejection yields numerous current steps of various magnitude. We analyze the step magnitudes for the downward-going current transitions because those are the steps that most likely result from the attachment of another peptide onto the particle. FIG. 5B shows the step-down distribution (FIG. 5B) overlaid on the previously established RGDC and GRGDSPC step transition distributions. The large peak centered at −6.8 pA nearly overlaps with the expected distribution from RGDC peptides, and we see a smaller number of events consistent with the GRGDSPC peptides centered at −9.7 pA. Clearly, the probability of attaching or exchanging a smaller peptide is greater than the larger peptide, which is not surprising given that the entropic penalty to bind a shorter peptide should be smaller. It is worth noting that the long times between current step transitions make peptide identification from a single current step straightforward. FIG. 5C illustrates this point where eight stepdown transitions are labeled with numbers that correspond to the different peptides involved. The numbers (steps 5, 6, and 8) most likely correspond to RGDC transitions, and the numbers (steps 2, 3, and 7) most likely correspond to GRGDSPC transitions. Steps 1 and 4 most likely correspond to a TP fluctuation. To quantify the likelihood of each step corresponding to a particular peptide, we performed a leastsquares fit using the RGDC and GRGDSPC distributions to the observed current step distribution. A complete description of the analysis can be found in the Nanopore Sensing and Data Analysis section.

Identifying the presence of various peptides from single capture events can also be extended to the high-frequency two state fluctuating peptides because the addition of each peptide is separated by several seconds. Given the fact that many of the so-called high-frequency peptides exhibit distinct signatures in their corresponding current histograms (see FIGS. 2 and 3), it should be possible to identify a particular peptide in a mixture. Analyzing high-frequency peptides for sensing applications requires an understanding of how fluctuations behave with the addition of multiple peptides. In the low frequency case (FIGS. 4 and 5), the addition of more than one peptide does not complicate the fluctuations. The same does not hold for the high-frequency case. To address this, we present an example of peptide mixture detection for 7C1 and 9C1 peptides.

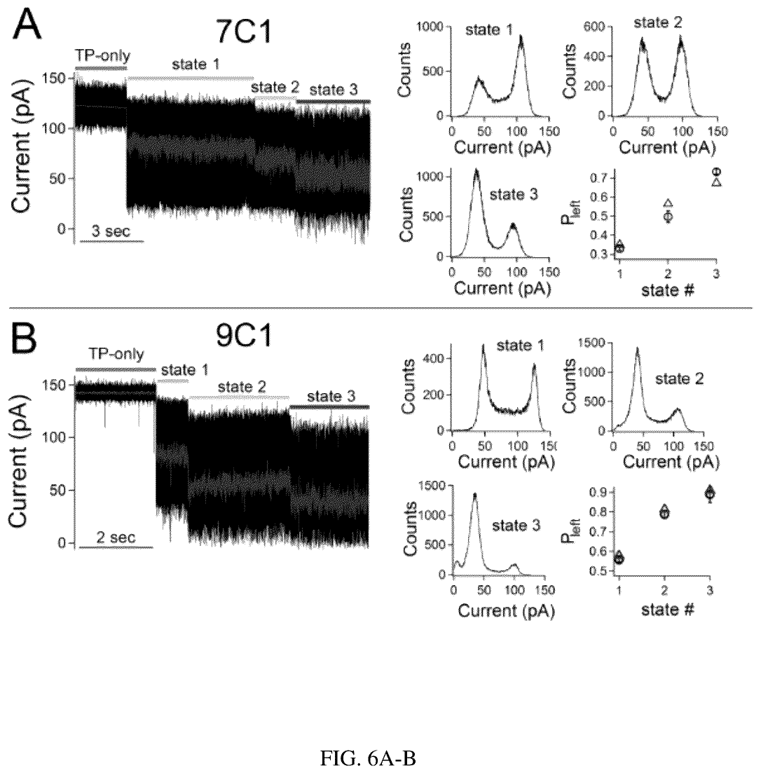

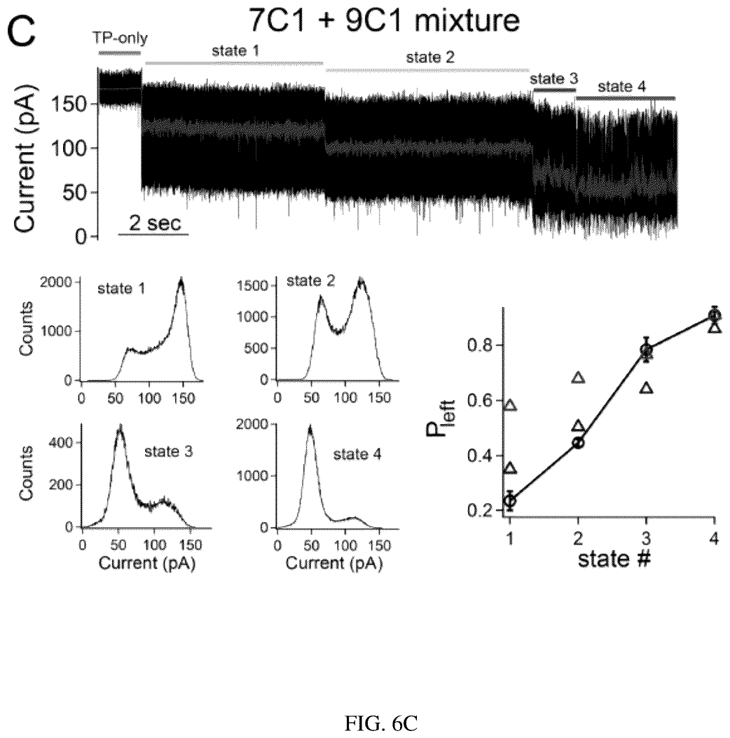

To begin, we show in FIG. 6A,B how current fluctuations change with the addition of sequential 7C1 and 9C1 peptides onto a trapped cluster. To analyze these events, we filter the current signal with a 100 Hz low-pass filter. This allows one to clearly identify when another peptide attaches to the cluster (as evidenced by a downward step in the filtered current). FIG. 6A shows the sequential addition of three 7C1 peptides on a single trapped cluster. The filtered data highlight each addition, and we calculate all-points histograms for each of the current states corresponding to the addition of another peptide. These distributions show a clear trend where the relative magnitude of the lower current state (with respect to the upper current state) increases with each additional peptide. This is most effectively parametrized by Pleft (see FIG. 2).

We developed a simple model for Pleft as a function of the number of peptides attached to the cluster. The model assumes the two-state current fluctuations are dependent on the peptides being in one of two conformations (see discussion connected to FIG. 3 for more details). If we assume each peptide's conformation state is independent of all the other peptides on the cluster, then we can calculate the probability of finding the current in the deeper state, and this is directly related to Pleft. Applying this model to the 7C1 data in FIG. 6A shows reasonable agreement with the observed fluctuations. Similar analysis is applied to the case of sequential 9C1 peptides attaching to the cluster. These results are highlighted in FIG. 6B.

In FIG. 6C, we present a typical current trace for a 7C1:9C1 mixture ejected onto a trapped cluster. In this case, we clearly identify four different states which we assume result from the addition of four peptides. Analyzing the Pleft parameter for each state and comparing these results to our model shows that the first two states most likely correspond to the addition of two 7C1 peptides and the last two states most likely correspond to the addition of two 9C1 peptides. This is evidenced by the fact that the Pleft data are in better agreement with the 7C1 model additions for states 1 and 2, and then the agreement is closer for 9C1 model additions for states 3 and 4. The overlap between the different peptides becomes difficult to resolve beyond state 3, which suggests that this Pleft model analysis is limited to 2 or 3 peptides per cluster.

Nanopore sensing with biological pores has been an active area of research for nearly 25 years. The technique provides a powerful analytical testbed with which to analyze unlabeled single molecule behavior in nanoconfined environments. Nanopore sensing has become increasingly focused on peptide detection in the past few years.⁴⁹ The cluster-based detection protocol outlined herein presents a number of advances for peptide detection. Cluster-based detection can selectively detect cysteine-containing peptides and remain blind to noncysteine containing peptides. This provides an advantage because a large percentage of peptides do not contain cysteine,⁵⁹ and this could serve as a selective tool to reduce the analytical complexity required to identify proteins from peptide fragments. While the cluster-based detection described herein may not yield distinct parameters for every peptide (see FIG. 4), it does give rise to very long-lived events (˜seconds), which could be used to optimize peptide mixture analysis to identify peptides that give rise to distinct signatures. The advantage here is that the gold cluster approach yields additional parameters (e.g., Pleft) beyond traditional open pore parameters (i.e., blockade depth, blockade time, etc.) that could be used to help identify a variety of cysteine containing peptides.

The long-lived fluctuations (like those highlighted in FIGS. 3 and 6) should yield useful information to help identify some peptide characteristics. Specifically, whether or not one or more peptides in a mixture contains a cysteine, where is the cysteine most likely positioned (near the N- or C-terminus), what is the approximate size of the peptide, and if the peptide is charged or neutral. All of this information, coupled with protein digestion from several cleavage enzymes,⁶⁰ could help identify a target peptide in a mixture.

There has been considerable development of parallel nanopore systems (i.e., Minion® from ONT) that are capable of high throughput analysis (ca. 100 s of individual nanopores), and we envision a similar setup could be used for our technique. Briefly, several microliters of gold clusters could be ejected with a pipet into the cis-side of the pore and microfluidic loading could be used to introduce and flush away peptide mixtures for continual analysis.⁶¹ The ability to apply independent voltages to a large number of nanopores would enable the application of voltage reversals to nanopores containing clusters with too many peptides attached. This would enable the detection of large numbers of peptide fluctuations from which one could ascertain the relative concentration of the cysteine-containing peptides in a mixture.

Alternatively, cluster-based analysis with a single pore may still prove useful for nanopore sensors because it could be used in conjunction with open pore analysis, which is more ideal for estimating peptide concentrations, to better inform the experimenter that an unknown peptide mixture contains a number of different cysteine-containing peptides. If one uses the clusters in addition to open pore analysis, then it could provide a powerful tool for improving the prospects for cysteine-containing peptide mixture analysis.

Open-pore resistive-pulse analysis is typically used to associate current blockades with a particular molecule.^{41,62} For the case of a simple polymer like PEG, this analysis is straightforward because the current blockades are low noise and single leveled.⁶³ Indeed, peptide analysis with open pores has had success for various homopolymer peptides⁶⁴ and transside analysis.⁶³ However, open pore analysis of peptides is generally more complicated because the peptides give rise to a wide variety of blockade types (short-lived, translocating, and multistate). These complex blockades result from a number of factors (e.g., solubility, secondary structure) that limits the pore's ability to distinguish between peptides. This can be seen from the open pore current traces and corresponding blockade examples of several of the peptides.

One can imagine an experiment where a peptide mixture is introduced to an open pore system. This gives rise to current blockades that may yield overlapping (low resolution) distributions among the different peptides in the mixture. The additional gold cluster data could help identify the presence of multiple cysteine-containing peptides. To illustrate this, we show a combination of open pore and cluster-based analysis of a 7C1 and 9C1 mixture. The open pore data result from ejection of the mixture onto an open αHL pore for an extended period (≈20 min). This gives rise to several thousand single molecule blockades whose various characteristics (i.e., blockade depth, duration, and standard deviation) are each distributed in a unimodal fashion. This means the open pore is essentially blind to the fact that there are two distinct peptides in the mixture. However, by measuring this same mixture with gold clusters in the pore, we find clear evidence of two distinct current signatures. This additional information correctly indicates the presence of two cysteine-containing peptides in the mixture, which do not appear in the open pore analysis.

Collecting gold cluster data, in parallel with the open pore data, could make it possible to identify the presence of multiple cysteine-containing peptides that may not be distinguishable from the open pore data alone. This information could better inform a machine learning search of peptide mixtures⁶⁵ and shows that our gold cluster protocol could be used for the analysis of unknown peptide mixtures.

### Conclusions

Nanopore-bound metallic clusters have shown promise as sensors via ligand exchange and surface attachment processes. Cysteine-containing peptides can be selectively detected, and the corresponding fluctuations can be used to identify each peptide attachment. Here, we have shown the different types of fluctuations that arise with the attachment of various peptides, and these yield either stable transitions between current states where the transition steps are well-correlated with the peptide mass or high-frequency two-state fluctuations. These high frequency fluctuations were shown to depend on various physical (length, charge) and chemical (sequence) characteristics. We then demonstrated that the peptide-induced fluctuations can be analyzed to identify peptides in mixtures from both the current-step and the high-frequency peptide types. In fact, the distinct nature of the high-frequency fluctuations provides a clear indication of the peptide that attaches at the single peptide limit. We believe this cluster-based approach to peptide sensing could provide a useful means for identifying low-mass cysteine-containing peptides that can play an important role in proteolytic-based peptide and protein sensors.

### Methods

Materials. Nanopore Experiments. 1,2-Diphytanoyl-sn-glycero-3-phospholcholine (DPhyPC) lipid was purchased from Avanti Polar Lipids (Alabaster, AL). Teflon sheets were purchased from Goodfellow USA Corp. (Coraopolis, PA). 50 μm diameter holes were formed in the Teflon sheets with laser drilling at Potomac Photonics (Halethrope, MD). Borosilicate glass capillaries, with filament, having an external diameter of 1 mm and internal diameters of 0.5 mm and 0.78 mm were purchased from Sutter Instruments (Novato, CA). The micropipettes for lipid ejection and for analyte ejection were made from the glass capillaries of 0.5 mm and 0.78 mm, respectively, using a P-2000 puller (Sutter Instruments) with preset program #11.

Chemicals such as potassium tetrachloroaurate (III) hydrate, Tris (hydroxymethyl) aminomethane (TRIS), tiopronin (TP), potassium chloride (KCl), hexadecane, citric acid, and potassium hydroxide (KOH) were purchased from Sigma-Aldrich (St. Louis, MO). Boranetert-butylamine complex (BTBC) was purchased from Alfa Aesar, (Ward Hill, MA). n-Pentane and methanol were purchased from Fisher Scientific (Washington, DC). Alpha toxin from Staphylococcus aureus was purchased from IBT Bioservices (Rockville, MD).

Peptide Synthesis and Purification. All chemicals and reagents used for peptide purification were purchased from Thermo-Fisher Scientific (Waltham, MA), VWR (Radnor, PA), or Sigma-Aldrich (St. Louis, MO).

Methodology. Cluster Synthesis. Water-soluble TP-capped gold clusters were synthesized using a previously established protocol.⁵¹ Solutions of potassium tetrachloroaurate (III) hydrate, TP, and BTBC were created at a concentration of 2.5 mM in methanol and then mixed in a 2:2:1 volumetric ratio, respectively. First, 700 UL of gold solution was mixed with 700 μL of TP ligand solution and shaken for several seconds. This was followed by the addition of 350 μL of BTBC solution. The final mixture was vortexed and sonicated for about 30 min during which time the solution would turn from clear to brown, indicating the formation of nanoclusters. This mixture was then left open in a fume hood for 12-24 h until all methanol evaporated away and the dried sample was affixed to the inside of the container surface. The dried sample was rehydrated using 1 mL of type I ultrapure water (18.2 M Ω·cm), and the sample was stored at 4° C. and remained stable over a period of 1-2 months. From the values listed here, we estimate the stock nanocluster concentration to be on the order of 20 μM (assuming an average of 100 gold atoms per cluster).

Peptide Synthesis and Characterization. Peptide variants were synthesized using standard FMOC chemistry in-house as previously described or purchased from GenScript (Piscataway, NJ).⁶⁶ Peptides were purified via reversed-phase HPLC Vydac C4 column, eluted by a linear gradient of water:acetonitrile (both supplemented with 0.1% TFA). Peptide identity and purity were confirmed using LC-MS to >95% purity. Peptide concentration was determined using Beer's law with an extinction coefficient of 5680 M-1 cm-1. Each peptide stock for CD analysis was made to a final concentration of 150-200 μM then stored at 4° C.

Nanopore Sensing and Data Analysis. The nanopore set up and methodology are nearly identical to one previously described.⁵¹ The electrolyte buffer used here was 3 M KCl, 10 mM Tris at pH 8.0. Following the formation of a single αHL channel in an unsupported DPhyPC membrane, we applied a 70 mV transmembrane voltage (Axopatch 200B, Molecular Devices, San Jose, CA), which yielded an easily detectable current through the nanopore. FIG. 1A illustrates the principle of operation where two microcapillaries are positioned ca. 50 μm from the pore-containing membrane, and a backing pressure (˜10 hPa) was applied for several seconds with a pump (Femtojet, Eppendorf, Hauppauge, NY) to eject TP-capped clusters into the cis-side of the αHL pore. After the capture of a single TPcapped cluster, the backing pressure was removed from the cluster-containing tip to eliminate the possibility of multiple cluster captures.

Following this capture, pressure was applied to a second capillary tip that contained the peptide of interest (˜10 s). This caused peptides to be ejected onto the pore-bound cluster, and current was recorded for extended periods during and after the peptide-tip pressure was applied. Unless stated otherwise, peptide concentration in the capillary tip was 500 μM and the total solution volume in each tip was ca. 5 μL. The peptide concentration at the pore is greatly reduced due to diffusion effects, and we estimate it to be on the order of 10 μM. Further discussion of this phenomenon can be found elsewhere.⁶⁷ To minimize effects from peptide aggregation at these higher concentrations, peptide solutions were typically discarded after 1 week.

Current signals were digitized and collected at a sampling rate of 50 kHz (Digidata 1550B, Molecular Devices) with a four-pole low-pass Bessel filter set to 10 kHz. Current traces were recorded with pCLAMP 10.7 software (Molecular Devices). Further analysis (i.e., histograms, digital filtering, and multipeak fitting) was performed with IGOR 6.37 (Wavemetrics, Portland, OR). Current signal traces were reported with either the 10 kHz filter or a postprocessed 100 Hz digital filter using IGOR software (4-pole, infinite impulse response filter). An in-house MATLAB-based (MATLAB R2019a, Mathworks, Natick, MA) cumulative sum analyzer was used to analyze current step distributions as needed.⁵⁶ All plots were generated using the IGOR software.

Percentage mixture analysis for the low-frequency peptides RGDC and GRGDSPC from FIG. 5 was performed as follows. Downward going current steps >4 pA were analyzed for 24 different exchange experiments (237 steps on 24 clusters over 3 different nanopores) and used to construct the step distributions, shown as the line with empty circles in FIG. 5A. This distribution was fit with two Gaussians (one for each peptide) f (i)=A exp(−((i−i0)/w)2). The following parameters were used to fit the distributions: (RGDC): A=38, i0=−6.75 pA, w=1.45 pA and (GRGDSPC): A=18, i0=−9.7 pA, w=0.75 pA. These distributions are represented with the (GRGDSPC) and (RGDC) transparent shaded functions in FIG. 5. The sum of the two distributions is the leastsquares fit to the experimental distribution. From this fitting, we arrive at functions that yield the probability of a current step (i) resulting from either a RGDC peptide PRGDC(i)=f RGDC(i)/(f RGDC(i)+f GRGDSPC(i)) or a GRGDSPC peptide PGRGDSPC(i)=f GRGDSPC(i)/(f RGDC(i)+f GRGDSPC(i)). These percentages are reported in the FIG. 5 caption.

TEM Measurements. The TP-Au nanoparticle images were obtained from a transmission electron microscope (TEM) (JEM-200, JEOL, Peabody, MA) operated at 200 keV and equipped with a cold cathode. Particles were ejected onto a mesh 200 carbon-coated Cu grid. Particle sizes were analyzed with ImageJ software in a manner previously described.⁶⁸

### Example 2. Cluster-Enhanced Nanopore Sensing of Ovarian Cancer Marker Peptides Abstract

Development of novel methodologies that can detect biomarkers of cancer or other diseases is both a challenge and a need for clinical applications. This partly motivates recent efforts related to nanopore-based peptide sensing. Our lab has focused on the use of gold nanoclusters for selective detection of cysteine-containing peptides. Specifically, tiopronin-capped gold nanoclusters, trapped in the cis-side of a wild type alpha hemolysin nanopore, provide a suitable anchor for the attachment of cysteine-containing peptides. It was shown that the attachment of these peptides onto a nanocluster yields unique current signatures that can be used to identify the peptide. In this Example, we apply this technique to the detection of ovarian cancer marker peptides ranging in length from 8 to 23 amino acid residues. It is found that sequence variability complicates the detection of low molecular weight peptides (<10 amino acid residues), but higher molecular weight peptides yield complex, high-frequency current fluctuations. These fluctuations are characterized with chi-squared and autocorrelation analysis that yield significantly improved selectivity when compared to traditional open-pore analysis. We also demonstrate the technique is capable of detecting the only two cysteine-containing peptides from LRG-1, an emerging protein biomarker, that are present in the urine of ovarian cancer patients. We further demonstrate detection of one of these LRG-1 peptides spiked into a sample of human female urine.

### Results

We analyzed 13 peptides and 5 fragments, that were selected from a database of peptides that uniquely present in the urine of ovarian cancer patients.²⁹ These peptides contain a single cysteine residue, which is necessary to facilitate attachment to the pore-bound gold nanocluster.²⁷ The nanopore opening and cluster are on the order of 2 nm³² so peptides were selected that range in length between n=8 and n=23 residues. The five peptide fragments are sequences that would result from trypsin or chymotrypsin digestion from some of the peptides.

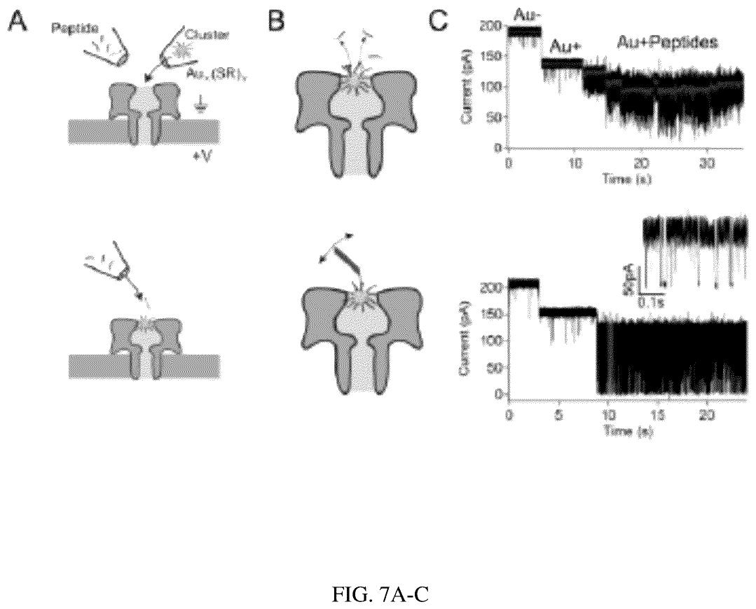

The principle of operation for the cluster-based nanopore detector has been previously described.^{27,30} FIG. 7 provides an illustration of the technique along with typical peptide-induced current traces. Briefly, a single, wild-type alpha-hemolysin pore is inserted into an artificial lipid bilayer membrane and two micropipette tips are positioned in proximity to the pore. Backing pressure is applied to the tip containing pre-formed gold nanoclusters until a single gold cluster is captured in the pore. After this, the gold-containing tip is removed and pressure is applied to the second tip containing the peptide of interest. This leads to peptide attachment to the gold cluster either through direct addition or ligand exchange.³¹ When small peptides (5-10 residues) are ejected onto the cluster, we see a number of current steps in the filtered signal (race FIG. 7C) corresponding to the addition of multiple peptides. Larger peptides (>10 residues) ejected onto the cluster typically yield a single attachment and long-lived fluctuating states.²⁷

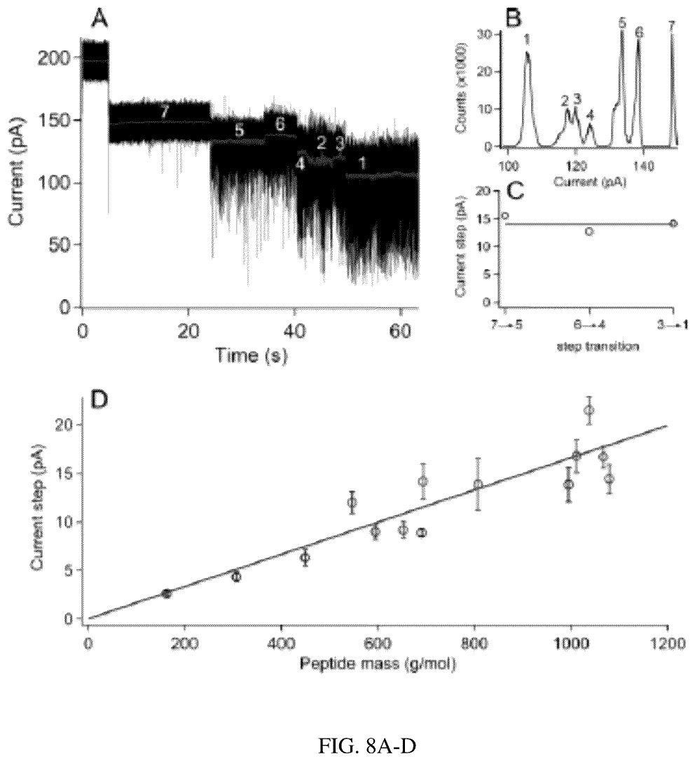

FIG. 8A highlights a typical current trace and corresponding all points histogram (FIG. 8B) of the filtered data (trace, FIG. 8A) from a so-called “low-frequency” peptide (P9C6). Numerous attachments of this peptide yield current steps whose magnitude are measured from the peak positions in FIG. 8B. Similar analysis for all the small peptides and fragments (P8C5, P9C6, P9C9, P9C6FC, P9C9FC, P16C1FT, P16C9FT, P16C15FT) yield the mean current step associated with each of these peptides. These mean values are reported as circles in FIG. 8D, and this data shows a generally linear dependence between peptide mass and current step magnitude. This is consistent with our previous study that analyzed current step magnitudes for a number of synthetic peptides and ligands.

While the trendline shows clear agreement between the synthetic and cancer-marker peptides, the scatter in the cancer marker peptides suggests that relying on the current step alone may not provide sufficient discrimination between different peptide sequences for sensing purposes. The increased scatter for the cancer marker peptides is not surprising given the random nature of the sequences studied herein as compared to the previously analyzed peptide sequences.²⁷ These stepwise fluctuations are also limited because each step only yields two pieces of information: the current step magnitude, which scales with the peptide mass,³³ and the duration of each current substrate, which scales with peptide concentration. Therefore, these low-frequency fluctuations may not be useful for unambiguously identifying the peptide. Nevertheless, this nanocluster-approach could still prove useful given the cysteine-selective detection provided by the cluster, but it is most likely that standard open-pore analysis will be more effective at detecting peptides in the smaller size range (n<10).

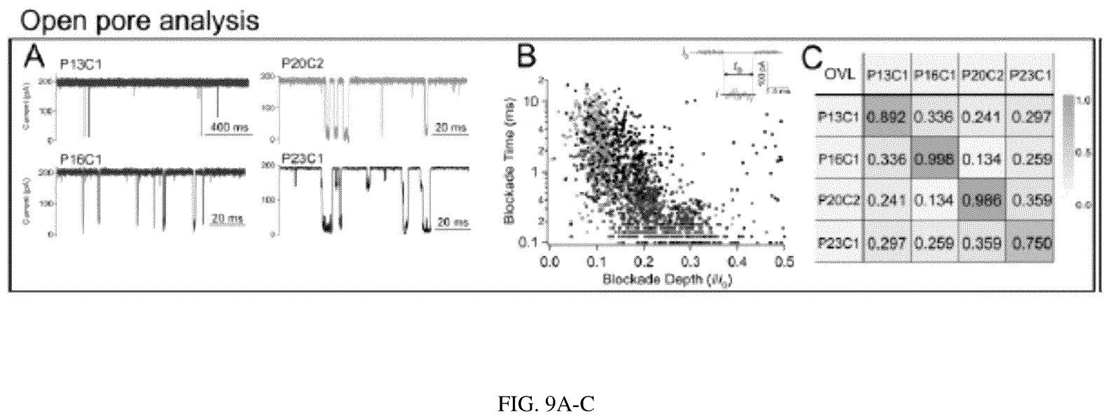

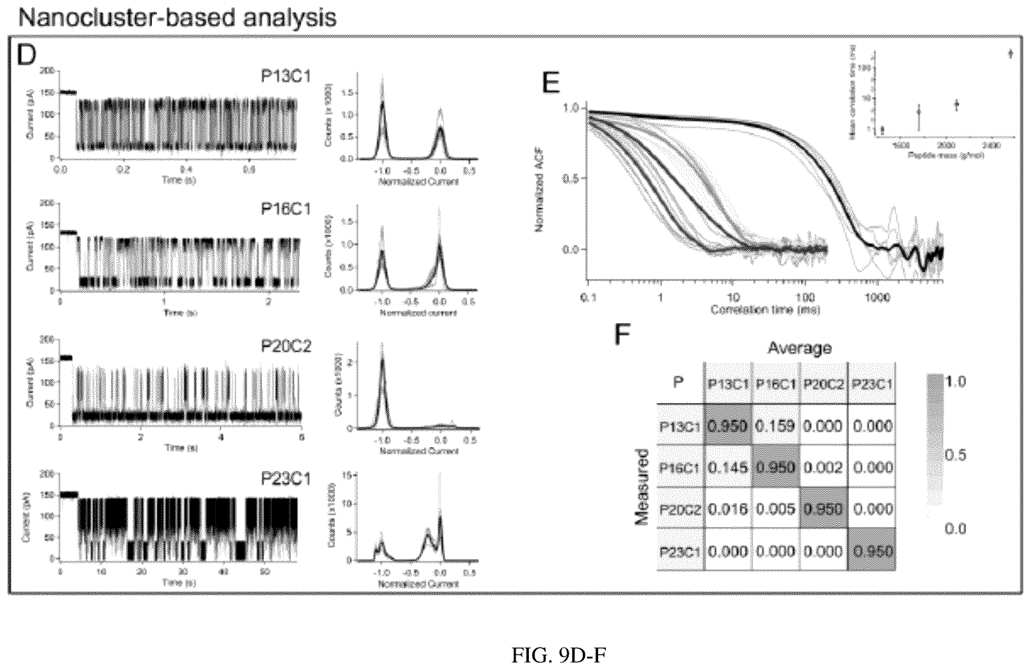

The advantage of our nanocluster-based approach becomes more obvious for larger peptides that give rise to so-called “high-frequency” fluctuations. We have previously shown²⁷ the attachment of larger peptides yields equal magnitude downward current steps, which suggests individual peptides can be bound to the cluster, overlaid with high-frequency two-state fluctuations. This enables analysis of individual peptides over extended periods, which give a more complete picture of the peptide due to its extended period of interaction on the cluster-modified nanopore. Motivated by this, we begin by analyzing four different-size peptides (P13C1, P16C1, P20C2 and P23C1) with the cysteine residue at or near the N-terminus of the sequence. Each of these peptides give rise to long-lived high-frequency fluctuations when analyzed with the nanocluster-occupied pore. We compare this to more traditional open pore analysis and show the nanocluster approach yields important advantages. FIG. 9A shows typical current traces in the open pore configuration with various current blockade signatures. The two smaller peptides yield short-lived downward-going spikes and the larger peptides show longer lived blockades with multistate structure. The charge-neutral peptide (P13C1) exhibits a far lower on-rate to the pore and thus required a different timescale to report several blockade events. The current blockades are all consistent with previous work that showed larger molecules spend more time in the pore.³⁴ This most likely results from increased enthalpic interactions between the peptide and nanopore.³⁵ Open-pore analysis typically compares the blockade depth (i/io) and blockade durations (tb) between different peptides as shown in FIG. 9B. Here we report 500 blockade events for each peptide, and we use the overlap percentage (FIG. 9C, overlap calculation described in data analysis subsection) to estimate the likelihood of incorrectly assigning a given blockade to the wrong peptide. For example, there is a 33.6% chance of identifying a P13C1 blockade as originating from a P16C1 peptide and vice-versa. The diagonal elements are less than one because the overlap was calculated in the range shown in FIG. 9B (0<i/io<0.5 and 0.1 ms<tb<20 ms), and some events fall outside this range (e.g. 89.2% of the P13C1 events are within the range shown in FIG. 9B). As can be seen from the off-diagonal elements, there is a considerable degree of overlap for peptides that differ in size over the reported range (i.e. 1400 g/mol-2600 g/mol) and this will complicate the open pore sensor's ability to correctly identify peptides in multicomponent mixtures.

The nanocluster-based detection improves the sensitivity of the nanopore in part because each molecule remains on the sensor for extended periods. FIG. 9D shows typical current traces where a gold nanocluster is captured by the pore and a peptide is subsequently attached to the cluster. High frequency fluctuations ensue, which yield complete details of the current distribution. Interestingly, the smaller peptides (P13C1 and P16C1) exhibit two-state fluctuations, which have been described previously²⁷ and larger peptides (P20C2 and P23C1) exhibit multistate fluctuations. These multistate fluctuations are suggestive of the peptide folding into different conformational states while bound to the nanocluster, and a previous study explored this in more detail for a different system.³⁶ Although free solution tryptophan fluorescence studies of the P23C1 peptide and circular dichroism spectra of both the P20C2 and P23C1 peptides show little evidence of secondary structure, it is possible that the cluster-pore environment and binding of the cysteine to the cluster lead to structural forms that are observable with the nanopore sensor that are not present in free solution. In any event, analysis of these multistate fluctuations and transition frequencies may serve to further improve discrimination between larger peptides. Here, we use both the current distribution and mean correlation times to more accurately identify each peptide in this collection.

To analyze nanocluster induced fluctuations, we used a chi-squared-based approach that calculates the probability of incorrectly identifying one peptide as another from the current histograms. We then calculated the autocorrelation function of each current trace to extract the mean and standard deviation of the correlation time for each peptide. From these results, we calculated the probability of measuring a given peptide and identifying it as a different peptide and reported that in the so-called P-matrix (probability) shown in FIG. 9F. More complete details describing these calculations can be found in the data analysis subsection. As an example of the meaning of the elements of the P-matrix, we find a 14.5% probability of measuring a P16C1 peptide and incorrectly identifying it as a P13C1 peptide. From FIG. 9F, it is clear that the nanocluster approach is more effective at correctly identifying these different-sized cancer marker peptides than the open pore analysis. It is also worth noting that the cluster-based approach is only sensitive to cysteine-containing peptides, which should greatly reduce the complexity of measuring peptide mixtures.

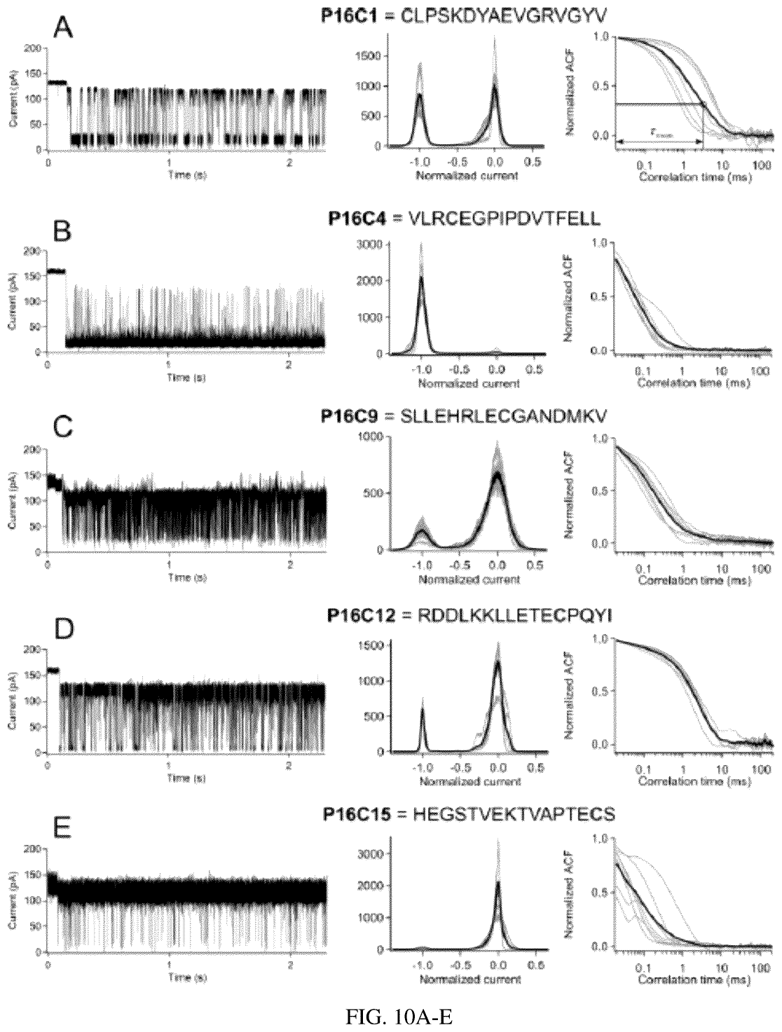

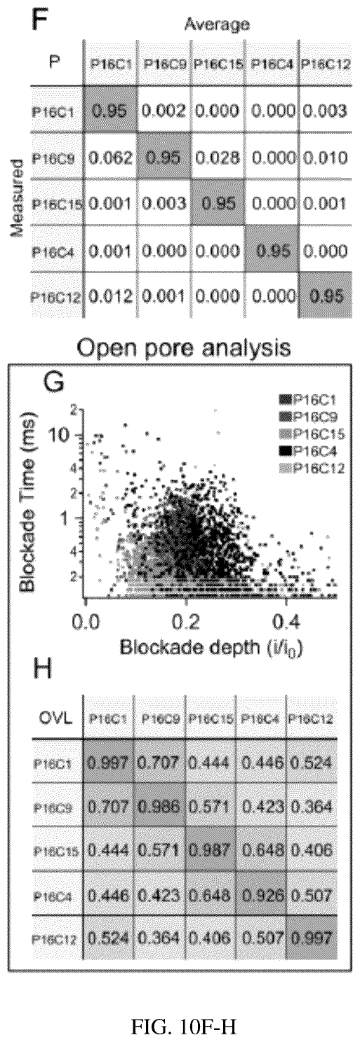

To further demonstrate the strength of the gold-cluster method we analyzed similar-size peptides where open pore fluctuations usually have a far more difficult time distinguishing between different molecules. FIG. 10 compares the nanocluster and open pore analysis for peptides P16C1, P16C4, P16C9, P16C12, and P16C15. Each peptide is 16 residues in length (and therefore approximately equal in mass (+/−106 amu)), but each has a different sequence and the cysteine residue is located at different positions along each sequence. FIG. 10A-E show sample current traces of the cluster-based detection of each peptide along with corresponding current histograms and normalized autocorrelation functions. From each current distribution and autocorrelation function we again calculate the detection probability matrix (P-matrix, FIG. 9F). Most of the off-diagonal elements are less than 1% with the largest probability (6.2%) corresponding to incorrectly identifying a measured P16C9 peptide as P16C1. This is in striking contrast to the overlap probabilities of the open pore blockades shown in FIGS. 10G-H where most peptides show a ca. 50% overlap. As already mentioned, the cluster-based approach also provides a considerable degree of selectivity because the gold clusters only yield signal from cysteine-containing peptides. This is amplified by the fact that cysteine residues are the naturally occurring amino acid with the lowest abundance in proteins, adding an additional layer of selectivity to the method. This will greatly reduce the complexity of detecting the numerous peptides that result from the proteolytic digestion of various proteins. As an important example of this for detecting cancer marker peptides, we explore the detection of peptides resulting from the digestion of leucine-rich α-2 glycoprotein 1 (LRG-1) proteins.³¹

LRG-1 has received increased attention for its role in many diseases. It has been directly connected with the onset and progression of eye, kidney, lung, heart and various inflammatory diseases. In addition, LRG-1 has been associated with a wide variety of cancers, including ovarian cancer.³¹ For the case of ovarian cancer, it was shown that LRG-1 peptides were only found in the urine of 6 ovarian cancer patients while only one LRG-1 peptide appeared in one of the 6 healthy (control) patients.²⁹ This suggests that detecting LRG-1 peptides could serve as an effective sensor for the onset of ovarian cancer.

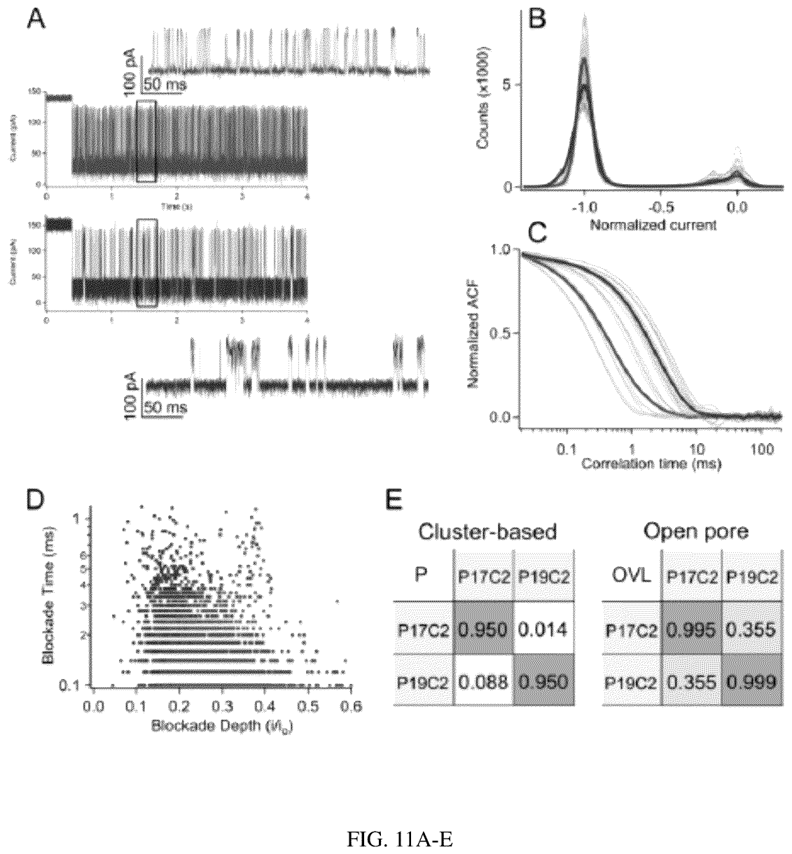

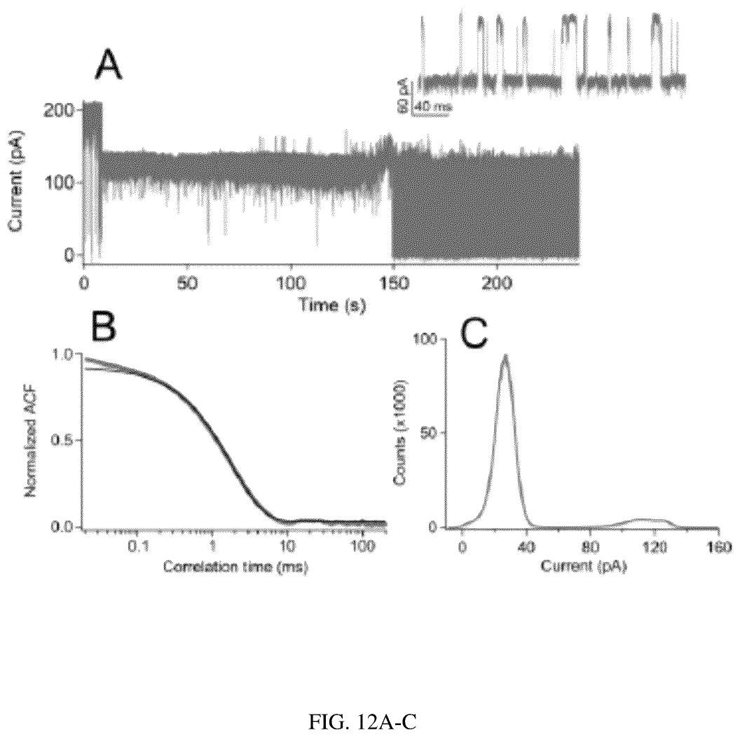

We demonstrate here that our nanocluster-based nanopore sensor could potentially detect for the presence of the LRG-1 protein. There are 90 different peptides originating from LRG-1 that are present in the urine of ovarian cancer patients.²⁹ FIGS. 11-12 show typical current traces and corresponding analysis of the only two cysteine-containing peptides from this collection of 90 (P17C2 and P19C2). The nanocluster-based analysis shows both peptides are detectable and yield clear differences in the high frequency fluctuating current traces. The chi-square and correlation time analysis yields a less than 10% likelihood of incorrectly identifying one for the other. We note that there is a 35.5% overlap from the open pore blockade analysis of these two peptides, but the nanocluster approach reduces the complexity of detecting LRG-1 because it is only sensitive to these two peptides. This reduction in detectable peptides should simplify signal analysis requirements.

### Conclusion

Nanopore sensing is becoming a viable, low-cost, and portable technique for single molecule detection and analysis. In particular, there is a growing interest in using nanopores for peptide detection. Typical measurements usually result in a large number of short-lived current blockades that yield limited bits of information (e.g. magnitude, duration and standard deviation) that can be used to identify the peptide. To improve selectivity, there has been a growing interest in exploring more sophisticated data analysis methods,³⁷ but there is still a need to improve the quantity and quality of data extracted from each peptide's interaction with the pore.

Here, we describe the application of a nanocluster-based approach for cysteine-selective peptide detection and we apply it to the detection of a number of ovarian cancer-marker peptides. For smaller peptides (<10 amino acid residues) we found a general trend between the current step magnitude and the peptide mass, but the limited data (one bit per peptide) and overall spread, that most likely results from sequence variability, calls into question the efficacy of the nanocluster technique for distinguishing between shorter peptides. However, for larger peptides, the nanocluster enables long interrogation times for single peptides and this yields far better discrimination when compared to more traditional open pore analysis methods. Importantly, the nanocluster approach was applied to the analysis of the only two cysteine-containing peptides that result from the highly sought LRG-1 protein biomarker in the ovarian cancer peptide library. This yielded clear discrimination between these two peptides, which suggests the nanocluster-based approach, applied to a parallel detection setup (e.g. MinION® from Oxford Nanopore Technologies) could provide a significant advancement for peptide detection and analysis.

### Methodology

### Materials:

1,2-diphytanoyl-sn-glycero-3-phosphocholine (DPhyPC) lipid was purchased from Avanti Polar Lipids (Alabaster, AL, USA). Alpha toxin from Staphylococcus aureus was purchased from IBT Bioservices (Rockville, MD, USA). Teflon supports for membrane formation were purchased from Goodfellow USA Corp. (Coraopolis, PA, USA). 50 um diameter holes were formed in the Teflon sheets with laser drilling at Potomac Photonics Inc. (Halethorpe, MD, USA). Borosilicate glass capillaries (with filament) were purchased from Sutter Instruments (Novato, CA, USA). Potassium tetrachloroaurate (III) hydrate, Tris, tiopronin, potassium chloride, hexadecane, citric acid, and potassium hydroxide were purchased from Sigma Aldrich (St. Louis, MO, USA). Borate-tert-butylamine complex was purchased from Alfa Aesar (Ward Hill, MA, USA). n-Pentane and methanol were purchased from Fisher Scientific (Washington, DC, USA). All peptides were purchased from GenScript (Piscataway, NJ, USA). All chemicals were used as received without further purification. Urine was collected from a female donor using informed consent in accordance with the approved Institutional Review Board Human Subjects Research Protocol (HM20002931). Urine was deposited into sterile collection cup supplied to the donor and returned on ice within 24 hours before aliquoting into 1 mL volumes and freezing at −80 C until use.

Fluorescence Spectroscopy: Samples were prepared in PBS adjusted to pH 8.0. Samples contained varying concentrations of peptide. Independent samples were prepared for each concentration. Fluorescence spectra were collected on a JY Fluoromax 4 with 2.5 nm slit widths. Fluorescence emission spectra from the tryptophan residues in the peptide were measured using 280 nm excitation wavelength and emission collected over the range of 300-400 nm. Background spectra from samples containing no peptide were subtracted from the sample spectra. Spectra were normalized such that the highest intensity was equal to 1.0. This calculation was also used to determine Xmax, the wavelength of highest intensity.

Circular Dichroism Spectroscopy: Circular Dichroism (CD) samples were prepared in PBS or in PBS supplemented with 3M KCl or 5M GuHCl. Samples contained 20 M peptide. CD spectra were collected on a JASCO CD Spectropolarimeter over the range 190-260 nm and each spectrum was an average of 64 scans. Background spectra from samples containing no peptide were subtracted from the sample spectra.

Nanoparticle Synthesis: This is achieved by a reduction of gold salts (KAuCl4) in the presence of a reducing agent as well as thiolated ligands, which in this case are Tiopronin(TP) and borane tert-butylamine complex (BTBC), Molar mixtures of (2:2:1) in Methanol solution. First 700 μL of gold salts and ligands are mixed and shaken vigorously for 30 seconds. Second 350 μL of BTBC is added and the resulting solution is vortexed for 30 seconds, after which it is sonicated for 30 minutes. During this time the solution will turn from a clear-yellow color to a darker amber color indicating the synthesis of the gold nanoparticles we desire. We then allow the particles to dry overnight under a fume hood for up to 24 hours and then the particles are resuspended in DI water and stored in a 4° C. refrigerator until used. According to previous works,³⁸ this synthesis should produce gold nanoparticles with little size variance and an average size of 2 nm. No further size exclusion is necessary because of the aforementioned size and distribution of particles but additionally the alpha hemolysin nanopore itself will exclude the translocation of any particles greater than 3 nm in size.³²

Nanopore Sensing: Studies exist providing great detail into the general methodology,³⁹ however our experimental setup remains virtually unchanged from our research groups previous publications.^{27,30} A lipid bilayer is formed across a 50 micron hole in a 20 micron thick teflon (PTFE) partition with DPhyPC through a modified painting method. Then a single alpha hemolysin channel is formed via the tip insertion method. The creation of a pore can be confirmed with change in current corresponding to the conductance of a single channel and proper orientation can also be confirmed by examining the current rectification and comparing against known values.⁴⁰ All peptide experiments were carried out in a buffer (3M KCL, 10 mM Tris, p.H=8.0).

A 1:4 nanoparticle:electrolyte solution was loaded into a borosilicate capillary (OD=1.0 mm and ID=0.78 mm) formed into a micropipette tip using preset program #11 on the P-2000 puller (Heat=350, Fil=4, Vel=30, Del=200) (Sutter Instruments, Novato, CA) with a final ID of 1 to 2 microns. This nanoparticle-filled micropipette was positioned ca. 50 microns above the membrane and ca. 20 microns to the left edge of the membrane on the cis-side of the pore (measured via the MPC-200 controller (Sutter)). A transmembrane voltage was applied (Axopatch 200B, Molecular Devices, San Jose, CA) with appropriate polarity (ground held fixed on cis-side of pore) and a backing pressure of approximately 15 hPa was applied through the tip to eject particles (Femtojet, Eppendorf, Hauppauge, NY). When a cluster is captured by the pore a noticeable drop in current occurs. The confirmation of a single particle capture is due to the fact the pore opening on the cis side is approximately 2.5 nm in size, therefore it is virtually impossible for multiple particles of 2 nm to fit into a single pore. To observe the exchange between peptides and the TP-Au-NP a second tip is prepared identically to the other and then filled with the peptide of choice. All peptide concentrations are 500 M unless otherwise stated. Urine sample was centrifuged for 5 minutes at xxxx rpm to reduce cellular and other organic debris from clogging tips. The solution was extracted from near the top of the aliquot. The second tip is then positioned symmetrically to the first at an equal distance from the pore on the opposite side. Then once a cluster is captured the pressure to the first tip is turned off and approximately 10 seconds are allowed to pass, then the pressure in the peptide containing tip is turned on. After some time spraying, exchange between the cluster and peptide can be observed giving rise to unique current signatures corresponding to the characteristics of the peptide in question (e.g size, charge, and sequence).

Data Processing: Current signals were digitized and collected at a sampling rate of 50 kHz (Digidata 1550B, Molecular Devices) with a four-pole low-pass Bessel filter set to 10 kHz. Current traces were recorded with pCLAMP 10.7 software (Molecular Devices). Further analysis (i.e., histograms, digital filtering, and multipeak fitting) was performed with IGOR 6.37 (Wavemetrics, Portland, OR). Current signal traces were reported with either the 10 kHz filter or a post processed 100 Hz digital filter using IGOR software (4-pole, infinite impulse response filter). An in-house MATLAB-based (MATLAB R2019a, Mathworks, Natick, MA) cumulative sum analyzer was used to analyze current step distributions as needed. All plots were generated using the IGOR software.

Analysis: Highfrequency open pore analysis: Open pore current blockades, like those shown in FIG. 9A, are analyzed with an in-house thresholding algorithm previously described.³⁴ The averaged open pore current, io, is calculated as the mean current before and after the onset of a blockade event. Blockade events are defined by current deviating by at least 3 standard deviations from the open pore current for at least 100 s. The averaged blockade current, i, is the average current between the down and up steps of the blockade event. The blockade time, tB, is the time between the up and down steps of the blockade event. The overlap probability (OVL(a,b)) matrix reported in FIG. 9C shows the probability of measuring a blockade from peptide type a and identifying it as peptide type b (e.g. the probability of measuring a blockade from P13C1 and identifying it as P16C1 is 33.6%). To calculate the ab element of the OVL matrix we divide the blockade times and blockade depths graph into N2 equally-sized regions and calculate the probabilities of finding peptide a and peptide b in each of the N2 regions. We then sum the minimum probability in each region over all N2 squares to calculate OVL(N). Finally, we calculate OVL(N) for N=10, 11, . . . ,20 and report the average value in the FIG. 9C matrix. The open pore data consists of 500 blockade events for each peptide, so we set an upper limit of N=20 so that on average, each square contains at least 1 blockade event. We note that the diagonal elements in the overlap matrix (OVL(a,a)) show the percentage of events for the a-type peptide that reside within the range of the graph shown in FIG. 9B (e.g. 89.2% of the blockade events for P13C1 fall within 0<i/io<0.5 and 0.1 ms<tB<20 ms).

Highfrequency cluster-based analysis: Cluster-captured peptides reside in the pore for extended periods which enables accurate characterization and discrimination that is superior to open pore analysis. Rather than identify various moments from each current distribution (mean, variance, etc.), we instead compare the overall current distributions directly with chi-square analysis. Additionally, we utilize autocorrelation functions to extract kinetic information, which can better identify a given peptide from its current trace.

We begin with the current distribution chi-square analysis. The current histograms in FIG. 9D and FIG. 10A-E are all calculated from 2-second current traces (the exception is P23C1 which were calculated from 20-second traces). To reduce event-by-event variability we shifted and normalized each current trace so the highest current state would correspond to 0 and the lowest current state would correspond to −1. To do this, we calculated a histogram from the unshifted/unnormalized current, then we performed multipeak fitting with Igor Pro software (Igor Pro 6.37, Wavemetrics, Portland, OR) to identify the highest probability upper and lower current levels. We then subtracted the highest current level from the current trace and divided the lowest current level from this shifted current trace. This resulted in a current trace that fluctuated between 0 and −1 and we refer to this as our “normalized current”, inorm. An 1100 bin histogram ranging from inorm=−1.4 to 0.667 with bin widths δ inorm=0.001875 was calculated for each shifted and normalized current trace. From these histograms, we calculated the average and standard deviation of each peptide's histogram from the arithmetic mean of each of the individual distributions.

To find the probability that a type-a peptide would be identified as a type-b peptide by comparison of the histograms, we calculated the reduced chi-square value for type-a peptides using the following formula,

$${χ}_{r , ( a , b )}^{2} = \frac{1}{{N}^{*}} \cdot {\sum }_{j = 1}^{1099} ( \begin{matrix}\frac{{( {H}_{a} ( {x}_{j} ) - {M}_{b} ( {x}_{j} ) )}^{2}}{{{SD}_{b} ( {x}_{j} )}^{2}} & if {M}_{b} ( {x}_{j} ) > {M}_{b , threshold} \\ 0 & if {M}_{b} ( {x}_{j} ) \leq {M}_{b , threshold}\end{matrix} ) \qquad ( 1 )$$

$${x}_{j} = - 1.4 + j δ {i}_{norm}$$

where Mb and SDb are the mean and standard deviation histogram values for the b-type peptide, and Ha is the histogram value of the a-type peptide. To reduce volatility in the calculation, we set a lower limit on the mean histogram value of Mb,threshold=50 (10 for P16C4 and P16C15 peptides) so N* is the number of bins in the average histogram where Mb>Mb,threshold.

Each a-type peptide yields a minimum of 5 different χ 2 r,(a,b) values from which we calculate an average and standard deviation for χ 2r,(a,b). Assuming a normal distribution of χ 2r,(a,b) values we calculated the probability that the measured χ 2r,(a,b) value would be less than 4 (p=0.05). This yields, Pₓ₂ (a,b) the probability that the a-type peptide fluctuations would be interpreted as originating from a b-type peptide assuming a 95% probability of assigning an a-type fluctuation to an a-type peptide.

To incorporate the kinetic information we measured the normalized autocorrelation functions for the same shifted and normalized current traces used for the chi-square analysis above. The normalized autocorrelation is defined by,

$$ACF ( τ ) = \frac{〈 I ( t ) I ( t + τ ) 〉 - {〈 I 〉}^{2}}{〈 {I}^{2} 〉 - {〈 I 〉}^{2}} \qquad ( 2 )$$

where <x> corresponds to the time average of the parameter x. This definition assumes a stationary process where t is not relevant. Given the rate of fluctuations (˜10 ms) versus the overall interrogation time (˜10 s) this is an acceptable approximation. From single exponential fits to the ACF we can extract the mean correlation time τmean. For each of the so-called high-frequency peptides, we measured at least 5 different events from which we find an average and standard deviation of τmean. Assuming a normal distribution of mean correlation times, we calculated overlap probabilities, P τ (a,b), for peptides of type a and b. Finally, assuming Pₓ₂ (a,b) and P τ (a,b) are independent and uncorrelated probabilities, we calculate the overall probability of identifying a blockade from peptide a as peptide b is given by,

$$P ( a , b ) = {P}_{{χ}^{2}} ( a , b ) {P}_{τ} ( a , b ) \qquad ( 3 )$$

This yields the matrix elements in FIG. 9F and FIG. 10F.

### REFERENCES FOR EXAMPLE 1

(1) Boschetti, E.; D'Amato, A.; Candiano, G.; Righetti, P. G. Protein Biomarkers for Early Detection of Diseases: The Decisive Contribution of Combinatorial Peptide Ligand Libraries. J. Proteomics 2018, 188, 1-14.
(2) Schulte, I.; Tammen, H.; Selle, H.; Schulz-Knappe, P. Peptides in Body Fluids and Tissues as Markers of Disease. Expert Rev. Mol. Diagn. 2005, 5, 145-157.
(3) Wang, X.; Yu, J.; Sreekumar, A.; Varambally, S.; Shen, R.; Giacherio, D.; Mehra, R.; Montie, J. E.; Pienta, K. J.; Sanda, M. G.; Kantoff, P. W.; Rubin, M. A.; Wei, J. T.; Ghosh, D.; Chinnaiyan, A. M. Autoantibody Signatures in Prostate Cancer. N. Engl. J. of Med. 2005, 353, 1224-1235.
(4) Chakraborty, S.; Andrieux, G.; Hasan, A. M. M.; Ahmed, M.; Hosen, M. I.; Rahman, T.; Hossain, M. A.; Boerries, M. Harnessing the Tissue and Plasma LncRNA-Peptidome to Discover Peptide-Based Cancer Biomarkers. Sci. Rep. 2019, 9, 12322.
(5) Frantzi, M.; van Kessel, K. E.; Zwarthoff, E. C.; Marquez, M.; Rava, M.; Malats, N.; Merseburger, A. S.; Katafigiotis, I.; Stravodimos, K.; Mullen, W.; Zoidakis, J.; Makridakis, M.; Pejchinovski, M.; Critselis, E.; Lichtinghagen, R.; Brand, K.; Dakna, M.; Roubelakis, M. G.; Theodorescu, D.; Vlahou, A.; Mischak, H.; Anagnou, N. P. Development and Validation of Urine-Based Peptide Biomarker Panels for Detecting Bladder Cancer in a Multi-Center Study. Clin. Cancer Res. 2016, 22, 4077-4086.
(6) Petricoin, E. F.; Belluco, C.; Araujo, R. P.; Liotta, L. A. The Blood Peptidome: A Higher Dimension of Information Content for Cancer Biomarker Discovery. Nat. Rev. Cancer 2006, 6, 961-967.
(7) van den Broek, I.; Sparidans, R. W.; Schellens, J. H. M.; Beijnen, J. H. Quantitative Assay for Six Potential Breast Cancer Biomarker Peptides in Human Serum by Liquid Chromatography Coupled to Tandem Mass Spectrometry. J. Chromatogr. B 2010, 878, 590-602.
(8) Cao, Z.; Kamlage, B.; Wagner-Golbs, A.; Maisha, M.; Sun, J.; Schnackenberg, L. K.; Pence, L.; Schmitt, T. C.; Daniels, J. R.; Rogstad, S.; Beger, R. D.; Yu, L.-R. An Integrated Analysis of Metabolites, Peptides, and Inflammation Biomarkers for Assessment of Preanalytical Variability of Human Plasma. J. Proteome Res. 2019, 18, 2411-2421.
(9) Murata, M.; Kawanishi, S. Oxidative DNA Damage Induced by Nitrotyrosine, a Biomarker of Inflammation. Biochem. Biophys. Res. Commun. 2004, 316, 123-128.
(10) Piktel, E.; Levental, I.; Durnas,' B.; Janmey, P. A.; Bucki, R. Plasma Gelsolin: Indicator of Inflammation and Its Potential as a Diagnostic Tool and Therapeutic Target. Int. J. Mol. Sci. 2018, 19, 2516.
(11) Al Shweiki, M. R.; Oeckl, P.; Pachollek, A.; Steinacker, P.; Barschke, P.; Halbgebauer, S.; Anderl-Straub, S.; Lewerenz, J.; Ludolph, A. C.; Bernhard Landwehrmeyer, G.; Otto, M. Cerebrospinal Fluid Levels of Prodynorphin-Derived Peptides Are Decreased in Huntington's Disease. Mov. Disord. 2021, 36, 492-497.
(12) Ashton, N. J.; Hye, A.; Rajkumar, A. P.; Leuzy, A.; Snowden, S.; Suirez-Calvet, M.; Karikari, T. K.; Schöll, M.; La Joie, R.; Rabinovici, G. D.; Höglund, K.; Ballard, C.; Hortobigyi, T.; Svenningsson, P.; Blennow, K.; Zetterberg, H.; Aarsland, D. An Update on Blood-Based Biomarkers for Non-Alzheimer Neurodegenerative Disorders. Nat. Rev. Neurol. 2020, 16, 265-284.
(13) Bibl, M.; Mollenhauer, B.; Lewczuk, P.; Esselmann, H.; Wolf, S.; Trenkwalder, C.; Otto, M.; Stiens, G.; Ru″ther, E.; Kornhuber, J.; Wiltfang, J. Validation of Amyloid-β Peptides in CSF Diagnosis of Neurodegenerative Dementias. Mol. Psychiatry 2007, 12, 671-680.
(14) Blankenberg, S.; McQueen, M. J.; Smieja, M.; Pogue, J.; Balion, C.; Lonn, E.; Rupprecht, H. J.; Bickel, C.; Tiret, L.; Cambien, F.; Gerstein, H.; Mu-nzel, T.; Yusuf, S. Comparative Impact of Multiple Biomarkers and N-Terminal Pro-Brain Natriuretic Peptide in the Context of Conventional Risk Factors for the Prediction of Recurrent Cardiovascular Events in the Heart Outcomes Prevention Evaluation (HOPE) Study. Circulation 2006, 114, 201-208.
(15) Braunwald, E. Biomarkers in Heart Failure. N. Engl. J. of Med. 2008, 358, 2148-2159.
(16) Magnussen, C.; Blankenberg, S. Biomarkers for Heart Failure: Small Molecules with High Clinical Relevance. J. Int. Med. 2018, 283, 530-543.
(17) Broza, Y. Y.; Zhou, X.; Yuan, M.; Qu, D.; Zheng, Y.; Vishinkin, R.; Khatib, M.; Wu, W.; Haick, H. Disease Detection with Molecular Biomarkers: From Chemistry of Body Fluids to Nature-Inspired Chemical Sensors. Chem. Rev. 2019, 119, 11761-11817.
(18) Machera, S. J.; Niedziólka-Jönsson, J.; Szot-Karpin′ska, K. Phage-Based Sensors in Medicine: A Review. Chemosensors 2020, 8, 61.
(19) Smith, C. R.; Batruch, I.; Bauc,a, J. M.; Kosanam, H.; Ridley, J.; Bernardini, M. Q.; Leung, F.; Diamandis, E. P.; Kulasingam, V. Deciphering the Peptidome of Urine from Ovarian Cancer Patients and Healthy Controls. Clin. Proteom. 2014, 11, 23.
(20) Begcevic, I.; Brine, D.; Brown, M.; Martinez-Morillo, E.; Goldhardt, O.; Grimmer, T.; Magdolen, V.; Batruch, I.; Diamandis, E. P. Brain-Related Proteins as Potential CSF Biomarkers of Alzheimer's Disease: A Targeted Mass Spectrometry Approach. J. Proteomics 2018,182, 12-20.
(21) Demirev, P. A.; Fenselau, C. Mass Spectrometry in Biodefense. J. Mass Spectrom. 2008, 43, 1441-1457.
(22) Macklin, A.; Khan, S.; Kislinger, T. Recent Advances in Mass Spectrometry Based Clinical Proteomics: Applications to Cancer Research. Clin. Proteom. 2020, 17, 17.
(23) Fricker, L. D. Limitations of Mass Spectrometry-Based Peptidomic Approaches. J. Am. Soc. Mass Spectrom. 2015, 26, 1981-1991.
(24) Dai, J.; Wang, J.; Zhang, Y.; Lu, Z.; Yang, B.; Li, X.; Cai, Y.; Qian, X. Enrichment and Identification of Cysteine-Containing Peptides from Tryptic Digests of Performic Oxidized Proteins by Strong Cation Exchange LC and MALDI-TOF/TOF MS. Anal. Chem. 2005, 77, 7594-7604.
(25) Eitner, K.; Koch, U.; Gawe, da, T.; Marciniak, J. Statistical Distribution of Amino Acid Sequences: A Proof of Darwinian Evolution. Bioinformatics 2010, 26, 2933-2935.
(26) Anfossi, L.; Baggiani, C.; Giovannoli, C.; D′Arco, G.; Giraudi, G. Lateral-Flow Immunoassays for Mycotoxins and Phycotoxins: A Review. Anal. Bioanal. Chem. 2013, 405, 467-480.
(27) Clerico, A.; Del Ry, S.; Giannessi, D. Measurement of Cardiac Natriuretic Hormones (Atrial Natriuretic Peptide, Brain Natriuretic Peptide, and Related Peptides) in Clinical Practice: The Need for a New Generation of Immunoassay Methods. Clin. Chem. 2000, 46, 1529-1534.
(28) Hage, D. S. Immunoassays. Anal. Chem. 1999, 71, 294-304.
(29) Hassanpour, S.; Hasanzadeh, M. Label-Free Electrochemical-Immunoassay of Cancer Biomarkers: Recent Progress and Challenges in the Efficient Diagnosis of Cancer Employing Electroanalysis and Based on Point of Care (POC). Microchem. J. 2021, 168, 106424.
(30) Li, Y.; Zhang, G.; Mao, X.; Yang, S.; De Ruyck, K.; Wu, Y. High Sensitivity Immunoassays for Small Molecule Compounds Detection—Novel Noncompetitive Immunoassay Designs. Trends Anal. Chem. 2018, 103, 198-208.
(31) Becker, J. O.; Hoofnagle, A. N. Replacing Immunoassays with Tryptic Digestion-Peptide Immunoaffinity Enrichment and LC-MS/MS. Bioanalysis 2012, 4, 281-290.
(32) Hoofnagle, A. N.; Wener, M. H. The Fundamental Flaws of Immunoassays and Potential Solutions Using Tandem Mass Spectrometry. J. Immunol. Methods 2009, 347, 3-11.
(33) Chang, P.-H.; Weng, C.-C.; Li, B.-R.; Li, Y.-K. An Antifouling Peptide-Based Biosensor for Determination of Streptococcus Pneumonia Markers in Human Serum. Biosens. Bioelectron. 2020, 151, 111969.
(34) Karimzadeh, A.; Hasanzadeh, M.; Shadjou, N.; Guardia, M. de la. Peptide Based Biosensors. Trends Anal. Chem. 2018, 107, 1-20.
(35) Liu, Q.; Wang, J.; Boyd, B. J. Peptide-Based Biosensors. Talanta 2015, 136, 114-127.
(36) Tian, L.; Heyduk, T. Antigen Peptide-Based Immunosensors for Rapid Detection of Antibodies and Antigens. Anal. Chem. 2009, 81, 5218-5225.
(37) Vanova, V.; Mitrevska, K.; Milosavljevic, V.; Hynek, D.; Richtera, L.; Adam, V. Peptide-Based Electrochemical Biosensors Utilized for Protein Detection. Biosens. Bioelectron. 2021, 180, 113087.
(38) Kasianowicz, J. J.; Robertson, J. W. F.; Chan, E. R.; Reiner, J. E.; Stanford, V. M. Nanoscopic Porous Sensors. Annu. Rev. Anal. Chem. 2008, 1, 737-766.
(39) Neuman, K. C.; Nagy, A. Single-Molecule Force Spectroscopy: Optical Tweezers, Magnetic Tweezers and Atomic Force Microscopy. Nat. Methods 2008, 5, 491-505.
(40) Zhao, Y.; Ashcroft, B.; Zhang, P.; Liu, H.; Sen, S.; Song, W.; Im, J.; Gyarfas, B.; Manna, S.; Biswas, S.; Borges, C.; Lindsay, S. Single-Molecule Spectroscopy of Amino Acids and Peptides by Recognition Tunnelling. Nat. Nanotechnol. 2014, 9, 466-473.
(41) Robertson, J. W. F.; Ghimire, M. L.; Reiner, J. E. Nanopore Sensing: A Physical-Chemical Approach. Biochimica et Biophysica Acta (BBA)—Biomembranes 2021, 1863, 183644.
(42) Brinkerhoff, H.; Kang, A. S. W.; Liu, J.; Aksimentiev, A.; Dekker, C. Multiple Rereads of Single Proteins at Single-Amino Acid Resolution Using Nanopores. Science 2021, 374, 1509-1513.
(43) Alfaro, J. A.; Bohlander, P.; Dai, M.; Filius, M.; Howard, C. J.; van Kooten, X. F.; Ohayon, S.; Pomorski, A.; Schmid, S.; Aksimentiev, A.; Anslyn, E. V.; Bedran, G.; Cao, C.; Chinappi, M.; Coyaud, E.; Dekker, C.; Dittmar, G.; Drachman, N.; Eelkema, R.; Goodlett, D.; Hentz, S.; Kalathiya, U.; Kelleher, N. L.; Kelly, R. T.; Kelman, Z.; Kim, S. H.; Kuster, B.; Rodriguez-Larrea, D.; Lindsay, S.; Maglia, G.; Marcotte, E. M.; Marino, J. P.; Masselon, C.; Mayer, M.; Samaras, P.; Sarthak, K.; Sepiashvili, L.; Stein, D.; Wanunu, M.; Wilhelm, M.; Yin, P.; Meller, A.; Joo, C. The Emerging Landscape of Single-Molecule Protein Sequencing Technologies. Nat. Methods 2021, 18, 604-617.
(44) Yan, S.; Zhang, J.; Wang, Y.; Guo, W.; Zhang, S.; Liu, Y.; Cao, J.; Wang, Y.; Wang, L.; Ma, F.; Zhang, P.; Chen, H.-Y.; Huang, S. Single Molecule Ratcheting Motion of Peptides in a Mycobacterium Smegmatis Porin A (MspA) Nanopore. Nano Lett. 2021, 21, 6703-6710.
(45) Zhang, S.; Huang, G.; Versloot, R. C. A.; Bruininks, B. M. H.; de Souza, P. C. T.; Marrink, S.-J.; Maglia, G. Bottom-up Fabrication of a Proteasome-Nanopore That Unravels and Processes Single Proteins. Nat. Chem. 2021, 13, 1192-1199.
(46) Huo, M.-Z.; Hu, Z.-L.; Ying, Y.-L.; Long, Y.-T. Enhanced Identification of Tau Acetylation and Phosphorylation with an Engineered Aerolysin Nanopore. Proteomics 2022, 22, 2100041.
(47) Versloot, R. C. A.; Lucas, F. L. R.; Yakovlieva, L.; Tadema, M. J.; Zhang, Y.; Wood, T. M.; Martin, N. I.; Marrink, S. J.; Walvoort, M. T. C.; Maglia, G. Quantification of Protein Glycosylation Using Nanopores. Nano Lett. 2022, 22, 5357-5364.
(48) Afshar Bakshloo, M.; Kasianowicz, J. J.; Pastoriza-Gallego, M.; Mathé, J.; Daniel, R.; Piguet, F.; Oukhaled, A. Nanopore-Based Protein Identification. J. Am. Chem. Soc. 2022, 144, 2716-2725.
(49) Robertson, J. W. F.; Reiner, J. E. The Utility of Nanopore Technology for Protein and Peptide Sensing. Proteomics 2018, 18, 1800026.
(50) Schmid, S.; StOmmer, P.; Dietz, H.; Dekker, C. Nanopore Electro-Osmotic Trap for the Label-Free Study of Single Proteins and Their Conformations. Nat. Nanotechnol. 2021, 16, 1244-1250.
(51) Cox, B. D.; Ghimire, M. L.; Bertino, M. F.; Reiner, J. E. Resistive-Pulse Nanopore Sensing of Ligand Exchange at the Single Nanocluster Limit for Peptide Detection. ACS Appl. Nano Mater. 2020, 3, 7973-7981.
(52) Fahie, M. A.; Chen, M. Electrostatic Interactions between OmpG Nanopore and Analyte Protein Surface Can Distinguish between Glycosylated Isoforms. J. Phys. Chem. B 2015, 119, 10198-10206.
(53) Nivala, J.; Mulroney, L.; Li, G.; Schreiber, J.; Akeson, M. Discrimination among Protein Variants Using an Unfoldase-Coupled Nanopore. ACS Nano 2014, 8, 12365-12375.
(54) Rostovtseva, T. K.; Gurney, P. A.; Hoogerheide, D. P.; Rovini, A.; Sirajuddin, M.; Bezrukov, S. M. Sequence Diversity of Tubulin Isotypes in Regulation of the Mitochondrial Voltage-Dependent Anion Channel. J. Biol. Chem. 2018, 293, 10949-10962.
(55) Xie, H.; Braha, O.; Gu, L.-Q.; Cheley, S.; Bayley, H. Single-Molecule Observation of the Catalytic Subunit of CAMP-Dependent Protein Kinase Binding to an Inhibitor Peptide. Chem. Biol. 2005, 12, 109-120.
(56) Cox, B. D.; Woodworth, P. H.; Wilkerson, P. D.; Bertino, M. F.; Reiner, J. E. Ligand-Induced Structural Changes of Thiolate-Capped Gold Nanoclusters Observed with Resistive-Pulse Nanopore Sensing. J. Am. Chem. Soc. 2019, 141, 3792-3796.
(57) Cox, B. D.; Martin, C. R.; Bertino, M. F.; Reiner, J. E. Biological Nanopores Elucidate the Differences between Isomers of Mercaptobenzoic-Capped Gold Clusters. Phys. Chem. Chem. Phys. 2021, 23, 7938-7947.
(58) Giesbers, M.; Kleijn, J. M.; Cohen Stuart, M. A. Interactions between Acid- and Base-Functionalized Surfaces. J. Colloid Interface Sci. 2002, 252, 138-148.
(59) Carugo, O. Amino Acid Composition and Protein Dimension. Protein Sci. 2008, 17, 2187-2191.
(60) Lucas, F. L. R.; Versloot, R. C. A.; Yakovlieva, L.; Walvoort, M. T. C.; Maglia, G. Protein Identification by Nanopore Peptide Profiling. Nat. Commun. 2021, 12, 5795.
(61) Fu, J.; Wu, L.; Qiao, Y.; Tu, J.; Lu, Z. Microfluidic Systems Applied in Solid-State Nanopore Sensors. Micromachines 2020, 11, 332.
(62) Reiner, J. E.; Balijepalli, A.; Robertson, J. W. F.; Campbell, J.; Suehle, J.; Kasianowicz, J. J. Disease Detection and Management via Single Nanopore-Based Sensors. Chem. Rev. 2012,112, 6431-6451.
(63) Chavis, A. E.; Brady, K. T.; Hatmaker, G. A.; Angevine, C. E.; Kothalawala, N.; Dass, A.; Robertson, J. W. F.; Reiner, J. E. Single Molecule Nanopore Spectrometry for Peptide Detection. ACS Sens. 2017, 2, 1319-1328.
(64) Piguet, F.; Ouldali, H.; Pastoriza-Gallego, M.; Manivet, P.; Pelta, J.; Oukhaled, A. Identification of Single Amino Acid Differences in Uniformly Charged Homopolymeric Peptides with Aerolysin Nanopore. Nat. Commun. 2018, 9, 966.
(65) Wan, Y. K.; Hendra, C.; Pratanwanich, P. N.; GOke, J. Beyond Sequencing: Machine Learning Algorithms Extract Biology Hidden in Nanopore Signal Data. Trends Genet. 2022, 38, 246-257.
(66) Kohn, E. M.; Shirley, D. J.; Arotsky, L.; Picciano, A. M.; Ridgway, Z.; Urban, M. W.; Carone, B. R.; Caputo, G. A. Role of Cationic Side Chains in the Antimicrobial Activity of C18G. Molecules 2018, 23, 329.
(67) Ghimire, M. L.; Gibbs, D. R.; Mahmoud, R.; Dhakal, S.; Reiner, J. E. Nanopore Analysis as a Tool for Studying Rapid Holliday Junction Dynamics and Analyte Binding. Anal. Chem. 2022, 94, 10027-10034.
(68) Schneider, C. A.; Rasband, W. S.; Eliceiri, K. W. NIH Image to ImageJ: 25 Years of Image Analysis. Nat. Methods 2012, 9, 671-675.

### References for Example 2 and Introduction

(1) Lei, Y.; Li, S.; Liu, Z.; Wan, F.; Tian, T.; Li, S.; Zhao, D.; Zeng, J. A Deep-Learning Framework for Multi-Level Peptide-Protein Interaction Prediction. Nat. Commun. 2021, 12 (1), 5465.
(2) Meyer, K.; Selbach, M. Peptide-Based Interaction Proteomics. Mol. Cell. Proteomics 2020, 19 (7), 1070-1075.
(3) Jiménez, M. Á.; González-Muniz, R. Peptides in Biology and Biomedicine: Walking towards the Future. Arch. Biochem. Biophys. 2019, 665, 20-22.
(4) Fujisawa, T.; Hayakawa, E. Peptide Signaling in Hydra. Int. J. Dev. Biol. 2012, 56 (6-7-8), 543-550.
(5) Lenaerts, C.; Monjon, E.; Van Lommel, J.; Verbakel, L.; Vanden Broeck, J. Peptides in Insect Oogenesis. Curr. Opin. Insect Sci. 2019, 31, 58-64.
(6) Marmiroli, N.; Maestri, E. Plant Peptides in Defense and Signaling. Peptides 2014, 56, 30-44.
(7) Cristiane R. Zuconelli, Roland Brock and Merel J. W. Adjobo-Hermans. Linear Peptides in Intracellular Applications. Curr. Med. Chem. 2017, 24 (17), 1862-1873.
(8) Pandey, S.; Malviya, G.; Chottova Dvorakova, M. Role of Peptides in Diagnostics. Int. J. Mol. Sci. 2021, 22 (16), 8828.
(9) Aydin, S. A Short History, Principles, and Types of ELISA, and Our Laboratory Experience with Peptide/Protein Analyses Using ELISA. Peptides 2015, 72, 4-15.
(10) Francisco Calderon-Celis, Jorge Ruiz Encinar, Alfredo Sand-Medel. Standardization Approaches in Absolute Quantitatve Proteomics with Mass Spectrometry. Mass Spectrom. Rev. 2018, 37 (6), 715-737.
(11) Neagu, A.-N.; Jayathirtha, M.; Baxter, E.; Donnelly, M.; Petre, B. A.; Darie, C. C. Applications of Tandem Mass Spectrometry (MS/MS) in Protein Analysis for Biomedical Research. Molecules 2022, 27 (8), 2411.
(12) Robertson, J. W. F.; Reiner, J. E. The Utility of Nanopore Technology for Protein and Peptide Sensing. PROTEOMICS 2018, 18 (18), 1800026.
(13) Varongchayakul, N.; Song, J.; Meller, A.; Grinstaff, M. W. Single-Molecule Protein Sensing in a Nanopore: A Tutorial. Chem. Soc. Rev. 2018, 47 (23), 8512-8524.
(14) Meyer, N.; Abrao-Nemeir, I.; Janot, J.-M.; Torrent, J.; Lepoitevin, M.; Balme, S. Solid-State and Polymer Nanopores for Protein Sensing: A Review. Adv. Colloid Interface Sci. 2021, 298, 102561.
(15) Hu, Z.-L.; Huo, M.-Z.; Ying, Y.-L.; Long, Y.-T. Biological Nanopore Approach for Single-Molecule Protein Sequencing. Angew. Chem. 2021, 133 (27), 14862-14873.
(16) Kennedy, E.; Dong, Z.; Tennant, C.; Timp, G. Reading the Primary Structure of a Protein with 0.07 Nm3 Resolution Using a Subnanometre-Diameter Pore. Nat. Nanotechnol. 2016,11 (11), 968-976.
(17) Ahmad, M.; Ha, J.-H.; Mayse, L. A.; Presti, M. F.; Wolfe, A. J.; Moody, K. J.; Loh, S. N.; Movileanu, L. A Generalizable Nanopore Sensor for Highly Specific Protein Detection at Single-Molecule Precision. Nat. Commun. 2023, 14 (1), 1374.
(18) Huang, G.; Voet, A.; Maglia, G. FraC Nanopores with Adjustable Diameter Identify the Mass of Opposite-Charge Peptides with 44 Dalton Resolution. Nat. Commun. 2019, 10 (1), 835.
(19) Cao, C.; Cirauqui, N.; Marcaida, M. J.; Buglakova, E.; Duperrex, A.; Radenovic, A.; Dal Peraro, M. Single-Molecule Sensing of Peptides and Nucleic Acids by Engineered Aerolysin Nanopores. Nat. Commun. 2019, 10 (1), 1-11.
(20) Zhang, X.; Galenkamp, N. S.; Van Der Heide, N. J.; Moreno, J.; Maglia, G.; Kjems, J. Specific Detection of Proteins by a Nanobody-Functionalized Nanopore Sensor. ACS Nano 2023,17 (10), 9167-9177.
(21) Zhao, Q.; Jayawardhana, D. A.; Wang, D.; Guan, X. Study of Peptide Transport through Engineered Protein Channels. J. Phys. Chem. B 2009, 113 (11), 3572-3578.
(22) Piguet, F.; Ouldali, H.; Pastoriza-Gallego, M.; Manivet, P.; Pelta, J.; Oukhaled, A. Identification of Single Amino Acid Differences in Uniformly Charged Homopolymeric Peptides with Aerolysin Nanopore. Nat. Commun. 2018, 9 (1), 966.
(23) Ensslen, T.; Sarthak, K.; Aksimentiev, A.; Behrends, J. C. Resolving Isomeric Posttranslational Modifications Using a Biological Nanopore as a Sensor of Molecular Shape. J. Am. Chem. Soc. 2022, 144 (35), 16060-16068.
(24) Huo, M.; Hu, Z.; Ying, Y.; Long, Y. Enhanced Identification of Tau Acetylation and Phosphorylation with an Engineered Aerolysin Nanopore. PROTEOMICS 2022, 22 (5-6), 2100041.
(25) Jiang, J.; Li, M.-Y.; Wu, X.-Y.; Ying, Y.-L.; Han, H.-X.; Long, Y.-T. Protein Nanopore Reveals the Renin-Angiotensin System Crosstalk with Single-Amino-Acid Resolution. Nat. Chem. 2023, 15 (4), 578-586.
(26) Lucas, F. L. R.; Versloot, R. C. A.; Yakovlieva, L.; Walvoort, M. T. C.; Maglia, G. Protein Identification by Nanopore Peptide Profiling. Nat. Commun. 2021, 12 (1), 5795.
(27) Ghimire, M. L.; Cox, B. D.; Winn, C. A.; Rockett, T. W.; Schifano, N. P.; Slagle, H. M.; Gonzalez, F.; Bertino, M. F.; Caputo, G. A.; Reiner, J. E. Selective Detection and Characterization of Small Cysteine-Containing Peptides with Cluster-Modified Nanopore Sensing. ACS Nano 2022, 16 (10), 17229-17241.
(28) Eitner, K.; Koch, U.; Gaweda, T.; Marciniak, J. Statistical Distribution of Amino Acid Sequences: A Proof of Darwinian Evolution. Bioinformatics 2010, 26 (23), 2933-2935.
(29) Smith, C. R.; Batruch, I.; Bauga, J. M.; Kosanam, H.; Ridley, J.; Bernardini, M. Q.; Leung, F.; Diamandis, E. P.; Kulasingam, V. Deciphering the Peptidome of Urine from Ovarian Cancer Patients and Healthy Controls. Clin. Proteomics 2014, 11 (1), 23.
(30) Cox, B. D.; Ghimire, M. L.; Bertino, M. F.; Reiner, J. E. Resistive-Pulse Nanopore Sensing of Ligand Exchange at the Single Nanocluster Limit for Peptide Detection. ACS Appl. Nano Mater. 2020, 3 (8), 7973-7981.
(31) Camilli, C.; Hoeh, A. E.; De Rossi, G.; Moss, S. E.; Greenwood, J. LRG1: An Emerging Player in Disease Pathogenesis. J. Biomed. Sci. 2022, 29 (1), 6.
(32) Song, L.; Hobaugh, M. R.; Shustak, C.; Cheley, S.; Bayley, H.; Gouaux, J. E. Structure of Staphylococcal α-Hemolysin, a Heptameric Transmembrane Pore. Science 1996, 274 (5294), 1859.
(33) Cox, B. D.; Woodworth, P. H.; Wilkerson, P. D.; Bertino, M. F.; Reiner, J. E. Ligand-Induced Structural Changes of Thiolate-Capped Gold Nanoclusters Observed with Resistive-Pulse Nanopore Sensing. J. Am. Chem. Soc. 2019, 141 (9), 3792-3796.
(34) Chavis, A. E.; Brady, K. T.; Hatmaker, G. A.; Angevine, C. E.; Kothalawala, N.; Dass, A.; Robertson, J. W. F.; Reiner, J. E. Single Molecule Nanopore Spectrometry for Peptide Detection. ACS Sens. 2017, 2 (9), 1319-1328.
(35) Angevine, C. E.; Robertson, J. W. F.; Dass, A.; Reiner, J. E. Laser-Based Temperature Control to Study the Roles of Entropy and Enthalpy in Polymer-Nanopore Interactions. Sci. Adv. 2021, 7 (17), eabf5462.
(36) Liu, S.-C.; Ying, Y.-L.; Li, W.-H.; Wan, Y.-J.; Long, Y.-T. Snapshotting the Transient Conformations and Tracing the Multiple Pathways of Single Peptide Folding Using a Solid-State Nanopore. Chem. Sci. 2021, 12 (9), 3282-3289.
(37) Das, N.; Mandal, N.; Sekhar, P. K.; RoyChaudhuri, C. Signal Processing for Single Biomolecule Identification Using Nanopores: A Review. IEEE Sens. J. 2021, 21 (11), 12808-12820.
(38) Bertino, M. F.; Sun, Z.-M.; Zhang, R.; Wang, L.-S. Facile Syntheses of Monodisperse Ultrasmall Au Clusters. J. Phys. Chem. B 2006, 110 (43), 21416-21418.
(39) Shi, W.; Friedman, A. K.; Baker, L. A. Nanopore Sensing. Anal. Chem. 2017, 89 (1), 157-188.
(40) Bhattacharya, S.; Muzard, J.; Payet, L.; Mathé, J.; Bockelmann, U.; Aksimentiev, A.; Viasnoff, V. Rectification of the Current in α-Hemolysin Pore Depends on the Cation Type: The Alkali Series Probed by Molecular Dynamics Simulations and Experiments. J. Phys. Chem. C 2011, 115 (10), 4255-4264.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of detecting cysteine-containing peptides, comprising
contacting a resistive-pulse nanopore sensing system with a sample containing cysteine-containing peptides of at least 10 amino acid residues in length, wherein the resistive-pulse nanopore sensing system comprises thiolate-capped gold clusters arranged in a cis side of nanopores; and
detecting a change in current indicative of exchange between a cysteine-containing peptide and the thiolate-capped gold clusters.

2. The method of claim 1, wherein the thiolate is tiopronin.

3. The method of claim 1, wherein the nanopores are protein nanopores.

4. The method of claim 3, wherein protein nanopores are alpha hemolysin nanopores.

5. The method of claim 4, wherein the alpha hemolysin is a wild-type alpha hemolysin.

6. The method of claim 1, wherein the sample is a biological sample obtained from a subject.

7. The method of claim 6, wherein the biological sample is selected from the group consisting of urine, blood, and saliva.

8. The method of claim 7, wherein the subject is suspected of having cancer and the cysteine-containing peptides are biomarkers of the cancer.


SEQUENCE LISTING (5 sequences)

SEQ ID NO: 1 - protein, 16 aa, synthetic construct
CLPSKDYAEVGRVGYV

SEQ ID NO: 2 - protein, 16 aa, synthetic construct
VLRCEGPIPDVTFELL

SEQ ID NO: 3 - protein, 16 aa, synthetic construct
SLLEHRLECGANDMKV

SEQ ID NO: 4 - protein, 16 aa, unidentified
RDDLKKLLETECPQYI

SEQ ID NO: 5 - protein, 16 aa, synthetic construct
HEGSTVEKTVAPTECS